(19) United States
(12) Patent Application Publication
(10) Pub. No.: US 2022/0049225 A1
(43) Pub. Date: Feb. 17, 2022

(54) CELL COMPOSITION COMPRISING LIVER PROGENITOR CELLS EXPRESSING HLA-E

(71) Applicant: PROMETHERA THERAPEUTICS SA, Mont-Saint-Guibert (BE)

(72) Inventors: Jean-Leon TCHELINGERIAN, Paris (FR); Elisa CORRITORE, Brussels (BE); Giuseppe MAZZA, London (GB); Nathalie BELMONTE, Nice (FR); Etienne SOKAL, Hoeilaart (BE)

(21) Appl. No.: 17/312,768

(22) PCT Filed: Dec. 12, 2019
(86) PCT No.: PCT/EP2019/084868

§ 371 Date: Jun. 10, 2021

(30) Foreign Application Priority Data
Dec. 14, 2018 (EP) PCT/EP2018/085024

(51) Int. Cl. C12N 5/071; A61K 35/407

(52) U.S. Cl. CPC C12N 5/0672; A61K 35/407; C12N 2501/231; C12N 2501/25; C12N 2501/2301; C12N 2501/24

(57) ABSTRACT

The current invention concerns isolated liver progenitor cells, cell lines thereof, cell populations comprising such and compositions comprising such wherein the liver progenitor cells are HLA-E positive. In addition, the invention concerns methods of preparing these liver progenitor cells.

### TECHNICAL FIELD

The invention pertains to the technical field of isolated liver progenitor cells or stem cells, originated from liver, and their use in medicine, hepatology, transplantation, liver failure and fibro-inflammatory liver diseases.

### BACKGROUND

Liver is a key organ performing many vital functions. Impairment of one of the multiple liver functions has a dramatic impact on health. Worldwide incidence of acute or chronic liver diseases sets these pathologies between the 5^{th} and the 9^{th} cause of death, according to the World Health Organization. Liver cell transplantation (LCT) is a procedure under clinical investigations for the treatment of hepatic diseases aiming at liver reparation/regeneration (Najimi et al., Stem Cells Translational Medicine, 2016). Despite the efforts to match the donor as immunologically close as possible to the recipient, issues arising after liver cell transplantation concerning rejection by the immune system remain a major issue after cell therapy, tissue transplant and specifically LCT. Mesenchymal stem cells (MSCs) are pleiotropic cells able to behave differentially depending on the extracellular environment especially those containing cytokines (Spees et al., Stem Cells research and therapy, 2016). Studies have shown that MSCs are hypo-immunogenic, can inhibit the development of an immune response, and skew diverse immune cell populations from pro-inflammatory towards anti-inflammatory/regulatory phenotypes (Najar et al., Inflammation research, 2018). At baseline levels, MSCs are minimally immunosuppressive. The cells can nevertheless adopt an immunosuppressive phenotype after exposure to specific environmental cues (Kouroupis et al., Tissue engineering Part B, 2018). Unfortunately, only a fraction of these naive MSCs become immunosuppressive after injection, depending on an individual patient's internal cues. For these reasons, there is a need to develop new MSC-phenotypes having improved immunosuppressive properties.

Keys to immune function and transplantation rejection are the major histocompatibility antigens, commonly referred to in humans as the HLA complex. Genes encoding class I HLA proteins are clustered at the telomeric end of human chromosome 6p21. These include the classical class Ia proteins, HLA-A, -B and -C, which are ubiquitously expressed, and are highly polymorphic. In contrast, the non-classical class Ib proteins, HLA-E, HLA-F and HLA-G, are relatively invariant, and are selectively expressed. The main function of HLA-class Ib molecules is to modulate the immune response by interacting with specific inhibitory receptors expressed on different immune effector cells, such as T- and B-lymphocytes, NK cells, and antigen-presenting cells.

MSCs derived from bone, cartilage, or adipose tissue have been described to express low levels of human leukocyte antigen (HLA) class Ia proteins. They may display immunomodulatory properties through the expression of soluble factors, including HLA-Ib proteins and in particular HLA-E (Morandi et al., Stem Cells, 2008). In physiological conditions, the expression of HLA-E is deeply linked to HLA-G (Morandi et al, Stem Cells, 2008; Morandi, Pistoia, Front. Immunol., 2014). HLA-E has been documented to be expressed in fetal human liver (Houlihan et al., 3. Immunol., 1992), adult hepatocytes and Kupffer cells (Araujo et al., 3. Immunol. Res., 2018). Such expression is increased after Hepatitis C virus (HCV) infection which suggests the potential immunomodulatory role of HLA-E in liver diseases (Araujo et al., 2018). Human induced pluripotent stem cell-derived retinal pigment epithelial cells constitutively express HLA-E which interaction with CD94/NKG2A receptor complex leads to a suppression of NK cells activation (Sugita et al., Invest. Ophthalmol. Vis. Sci., 2018). After bone marrow transplantation, HLA-E has been shown to be associated with lower risk of graft-versus-host-disease and decreased mortality (Pabon et al., Transplant Proceedings 2014). In vitro priming of adipose tissue MSC with IFN-γ increases the expression of HLA-E and other immunosuppressive proteins to potentialize the T cell inhibition (Wobma et al., 3. Immunol. Regen. Med., 2018).

WO2008121894 describes a cellular composition of MSCs having an enriched population of HLA-E positive cells, or HLA-E positive cells, or both HLA-E and HLA-G positive cells, and their potential use in the treatment of degenerative diseases and the immunomodulation of transplantation. WO2018081514 describes immunosuppressive mesenchymal stromal cells which are obtained by applying a proinflammatory cytokine to mesenchymal stromal cells in a hypoxic culture condition in vitro. US20120328578 discloses a method of treating inflammation in a synovial joint based on the induction of immunosuppressive properties in stem cells by treatment of the cells with synovial fluid of inflamed joint or with pro-inflammatory cytokines.

ADHLSCs and HHALPCs (also referred as HALPC, Human Allogeneic Liver Progenitor Cell) produced at higher scale for pursuing clinical studies, generated under GMP conditions) are undifferentiated progenitor cells obtained after collagenase digestion of the normal adult liver and primary culture of the parenchymal fraction. HHALPCs display self-renewing capacity and have the particularity to express both mesenchymal and hepatocytic markers and to display advanced hepatogenic differentiation potential. These cells are of particular interest in targeting human liver diseases, including liver fibrosis, Non-Alcoholic Steatohepatitis (NASH) and acute-on-chronic liver failure (ACLF), and other human diseases.

Raicevic et al. (Cytotherapy, 2015) discloses a method wherein adult-derived human liver mesenchymal stromal cells (ADHLSCs) are supplemented with an inflammatory cytokine cocktail overnight. No membranous and intracellular expression of HLA-E or HLA-G was observed. Raicevic also stays silent on the presence of a composition having a high amount of HLA-E expressing cells.

Mehdi Najar et al., 2018 discloses inflammatory primed ADHLSCs. This document does not mention the induction of HLA-E, nor the expression of HLA-G in the inflammatory primed ADHLSCs. Mehdi Najar stays completely silent on the benefits that may be linked to HLA-E expressing cellular compositions.

Hoda El-Kehdy et al., 2017 also discloses inflammatory primed ADHLSCs and shows that the expression of HLA-ABC, HLA-DR and HLA-G in (un)differentiated ADHLSCs in constitutive and primed state, showing no significantly induced expression of HLA-G or HLA-DR expression.

None of these prior art documents disclose the need to obtain a HLA-E expressing population of liver stem or progenitor cells for use in the clinic, nor do they mention that sufficient HLA-E expression might play a role in escaping cytolysis.

There remains a need in the art for new or improved allogeneic cell therapies with immunomodulatory as well as enhanced immunosuppressant and/or immunoevasive activity, for example for the prevention or the treatment of inflammation, auto-immune diseases and graft rejection, especially for the treatment of pathologies related to liver diseases.

### SUMMARY OF THE INVENTION

The present invention provides a composition comprising human adult liver-derived progenitor or stem cells according to claim 1, an isolated liver progenitor cell or stem cell according to claim 8 and a cell population according to claim 21. More in particular, the invention provides cells which are HLA-E positive and compositions comprising at least 60% of such HLA-E positive cells. The observed HLA-E expression in cells of the invention is mainly if not predominantly on the cell surface. It was found that compositions having an increased amount of HLA-E expressing cells (on their cell surface) were able to escape CD8+ T-cell mediated cytolysis and thus are especially suited for use in therapy.

The HLA-E expression in the cells, cell populations and compositions comprising said cells of the current invention is shown to considerably enhance its immunosuppressive and/or immunoevasive properties. These cells, cell populations and compositions are particularly well suited for use in the treatment or prevention of disorders involving unwanted immune responses, for example, but not limited to inflammation, auto-immune disease and graft rejection, as well as for use in the treatment of liver diseases.

The isolated liver progenitor cells, cell populations or compositions comprising said cells maintain their proliferation capacity, which together with their liver specificity lead to an increased efficacy and safety for liver cell transplantation. Furthermore, because of their adult origin, these stem cells obviate the immunological, ethical and carcinogenic issues associated to embryonic cells (Volarevic et al., International Journal of Medical sciences, 2018).

The enhanced immunosuppressive character of the progenitor cells owing to HLA-E expression contributes to decrease the immune-response related risks of cell therapy and/or tissue transplant, which are particularly high during allogeneic cell transplantations. Accordingly, the HLA-E positive liver-derived progenitor cells of the invention are more likely to be successful as a cell therapy for multiple liver disorders with improved efficacy and tolerability for the patient.

In a further embodiment of the invention, the liver progenitor cells, cell populations or compositions comprising said cells can also be positive for HLA-G or can have an elevated level of HLA-G expression. It is deemed that the co-operation of HLA-E and HLA-G may further enhance the immunosuppressive action of the respective liver progenitor cells, making them better suited for use in cell therapy approaches (Morandi, Pistoia, Frontiers in Immunology 2014).

In another further embodiment of the invention, the liver-derived progenitor cells, cell populations or compositions comprising said cells further are positive for at least one mesenchymal marker. Mesenchymal markers include but are not limited to Vimentin, CD13, CD90, CD73, CD44, CD29, a-smooth muscle actin (ASMA) and CD140-b. In another further embodiment, the liver-derived progenitor cells of the invention secrete HGF. In another further embodiment the liver-derived progenitor cells of the invention are negative for HLA-DR. In a further embodiment, the liver-derived progenitor cells are optionally positive for at least one hepatic marker and/or have optionally at least one liver-specific activity. Hepatic markers include but are not limited to albumin (ALB), HNF-3B, HNF-4, CYP1A2, CYP2C9, CYP2E1, CYP3A4 and alpha-1 antitrypsin. Liver-specific activities include but are not limited to urea secretion, bilirubin conjugation, alpha-1-antitrypsin secretion and CYP3A4 activity.

In a second aspect, the present invention provides a method of preparing HLA-E positive liver progenitor cells or stem cells according to claim 30. More in particular, the method comprises the step of preconditioning the cells which refers to adding one or more cytokines to the cell medium of a culture of liver progenitor or stem cells.

In a preferred embodiment the composition of the invention is suitable for use according to claims 24 to 29. More in particular the composition of the invention is provided for use in the treatment of a number of diseases where its immunosuppressive properties are especially beneficial.

In another aspect, the present specification also describes a method for the treatment and/or prevention of disorders and diseases where the immunosuppressive properties of the cells, cell populations and/or compositions as disclosed herein are particularly beneficial, including disorders with unwanted immune responses, fibrotic disorders and liver diseases. Unwanted immune responses include for example, but are not limited to inflammation, auto-immune diseases and graft rejection.

More in particular the invention provides a method, comprising the administration of a progenitor cell or stem cell, cell line thereof, cell population and/or composition of the invention to a subject in need of such treatment. Such administration is typically in therapeutically effective amount, i.e., generally an amount which provides a desired local or systemic effect and performance.

In yet another aspect, the present disclosure also describes the use of a progenitor cell or stem cell, cell line thereof, cell population and/or composition of the invention for use in therapy and/or for use in the manufacture of a medicament for the treatment and/or prevention of disorders with unwanted immune responses, for the treatment and/or prevention of fibrotic disorders, as well as for the treatment and/or prevention of liver diseases. Such diseases may include disorders affecting liver tissue, diseases affecting the hepatocyte viability and/or function as well as chronic liver failure caused by fibro-inflammatory reactions. In addition, owing to the immunomodulatory and immunosuppressive properties the use of a progenitor cell or stem cell, cell line thereof, cell population and/or composition of the invention is provided for use in therapy and/or for use in the manufacture of a medicament for the treatment of diseases wherein undesired immune responses are to be avoided.

### BRIEF DESCRIPTION OF FIGURES

FIG. 1, FIG. 2 and FIG. 3 show HLA-E expression of liver progenitor cells according to various embodiments of the invention.

FIG. 1 presents FACS analysis data showing HLA-E protein expression in different culture conditions for liver progenitor cells isolated from one donor (VLK019) compared to untreated control condition: IL-1β/TNFα/IFNγ in combination, IL-10 alone and IL-1β/TNFα/IFNγ/IL-10 in combination (“All together”). The green histogram ‘ISO’ is the isotype control (baseline, no fluorescence detected, no HLA-E expression detected).

FIG. 2 presents FACS analysis data showing HLA-E protein expression in different culture conditions for liver progenitor cells isolated from three different donors compared to untreated control condition: IL-1β/TNFα/IFNγ in combination, IL-10 alone and IL-1β/TNFα/IFNγ/IL-10 in combination (“All together”). The green histogram is the isotype control (baseline, no fluorescence detected, no HLA-E expression detected).

FIG. 3 shows FACS analysis data showing Mean Fluorescence Intensity (n=3), for HLA-E expression, detected in different culture conditions compared to untreated control condition: IL-1β/TNFα/IFNγ in combination, IL-10 alone and IL-1β/TNFα/IFNβ/IL-10 in combination (“All together”). An elevated level of HLA-E expression in preconditioned cells compared to untreated cells is confirmed.

FIG. 4 and FIG. 5 show gene expression profiles of HLA-E positive liver progenitor cells according to various embodiments of the invention.

FIG. 4 presents RT-PCR data showing fold increase in the gene expression of HLA-E and HLA-G in three different culture conditions compared to untreated control condition (baseline): IL-1β/TNFα/IFNγ in combination, IL-10 alone and IL-1β/TNFα/IFNγ/IL-10 in combination (“All together”).

FIG. 5 presents RT-PCR data showing fold increase in the gene expression of IDO1, HGF, PTGS2, IL-6 and IL-10 in preconditioned liver progenitor cells according to embodiments of the invention as compared to the untreated control (baseline). Increased gene expression levels for IDO1, PTGS2, IL-6 and/or IL-10 were confirmed when cells were pre-treated with pro-inflammatory cytokines selected from IFNγ, TNFα, IL-1β, IL-10 or combinations thereof.

FIG. 6 shows quantification of HLA-G protein secreted by isolated liver progenitor cells which are HLA-E positive according to an embodiment of the invention.

FIG. 6 presents secretion analysis data showing detectable levels of soluble HLA-G in supernatants recovered from different culture conditions: untreated (control condition), treated with IL-1β/TNFα/IFNγ in combination, IL-10 alone or IL-1β/TNFα/IFNγ/IL-10 in combination (“All together”). Human melanoma Bowes cells (HMBCs), transfected with a plasmid carrying the human HLA-G sequence, were used as positive control (“HMBCs Transfected”). These data confirm elevated HLA-G secretion by preconditioned isolated liver progenitor cells which are positive for HLA-E expression according to embodiments of the invention.

FIG. 7 shows secretion of immunoregulatory factor PGE2 in a cell population comprising HLA-E positive liver progenitor cells in various embodiments of the invention.

FIG. 7 presents secretion analysis data showing detectable levels of PGE2 in supernatants from different conditions including untreated liver progenitor cells (control condition) and liver progenitor cells which have been preconditioned with IL-1β/TNFα/IFNγ in combination, IL-10 alone or with IL-1β/TNFα/IFNγ/IL-10 in combination (“All together”). Enhanced secretion levels were observed in the preconditioned cells according to embodiments of the invention, as compared to the untreated condition.

FIG. 8 shows elevated IDO1 enzymatic activity levels in a cell population comprising HLA-E positive liver progenitor cells in various embodiments of the invention.

Data obtained in an enzymatic assay showing detectable levels of IDO activity in supernatants from different experimental conditions is presented in FIG. 8. The different conditions include untreated liver progenitor cells (control condition) and liver progenitor cells which have been preconditioned with IL-1β/TNFα/IFNγ in combination, IL-10 alone or with IL-1β/TNFα/IFNγ/IL-10 in combination (“All together”). Enhanced IDO activity was observed in the preconditioned cells according to embodiments of the invention.

FIGS. 9A, 9B and 9C show HLA-E expression in a population of liver progenitor cells before and after conditioning.

FIGS. 10A, 10B, 10C and 10D show the effect of cell compositions and cells according to the current invention on CD8 T-cell mediated cytolysis.

### DETAILED DESCRIPTION OF THE INVENTION

The present invention concerns isolated liver-derived progenitor cells or stem cells which are HLA-E positive, a method of preparing such cells, compositions comprising such cells and their use for the treatment or prevention of disorders involving unwanted immune responses, such as inflammation, auto-immune disease and graft rejection, as well as for the treatment of liver disease.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

The recitation of numerical ranges by endpoints includes all numbers and fractions subsumed within that range, as well as the recited endpoints.

The expression “% by weight”, “weight percent”, “% wt” or “wt %”, here and throughout the description unless otherwise defined, refers to the relative weight of the respective component based on the overall weight of the formulation.

As used herein, the term “isolated cell” refers generally to a cell that is not associated with one or more cells or one or more cellular components with which the cell is associated in vivo. For example, an isolated cell may have been removed from its native environment, or may result from propagation, e.g., ex vivo propagation, of a cell that has been removed from its native environment.

The term “in vitro” as used herein denotes outside, or external to, animal or human body. The term “in vitro” as used herein should be understood to include “ex vivo”. The term “ex vivo” typically refers to tissues or cells removed from an animal or human body and maintained or propagated outside the body, e.g., in a culture vessel.

The term “liver progenitor cell” refers to an unspecialized and proliferation-competent cell which is produced by culturing cells that are isolated from liver and which or the progeny of which can give rise to at least one relatively more specialized cell type. A liver progenitor cell give rise to descendants that can differentiate along one or more lineages to produce increasingly more specialized cells (but preferably hepatocytes or hepato-active cells), wherein such descendants may themselves be progenitor cells, or even to produce terminally differentiated liver cells (e.g. fully specialized cells, in particular cells presenting morphological and functional features similar to those of primary human hepatocytes). The term “stem cell” refers to a progenitor cell capable of self-renewal, i.e., can proliferate without differentiation, whereby the progeny of a stem cell or at least part thereof substantially retains the unspecialized or relatively less specialized phenotype, the differentiation potential, and the proliferation competence of the mother stem cell. The term encompasses stem cells capable of substantially unlimited self-renewal, i.e., wherein the capacity of the progeny or part thereof for further proliferation is not substantially reduced compared to the mother cell, as well as stem cells which display limited self-renewal, i.e., wherein the capacity of the progeny or part thereof for further proliferation is demonstrably reduced compared to the mother cell.

Based on the ability to give rise to diverse cell types, a progenitor or stem cell may be usually described as totipotent, pluripotent, multipotent or unipotent. A single “totipotent” cell is defined as being capable of growing, i.e. developing, into an entire organism. A “pluripotent” cell is not able of growing into an entire organism, but is capable of giving rise to cell types originating from all three germ layers, i.e., mesoderm, endoderm, and ectoderm, and may be capable of giving rise to all cell types of an organism. A “multipotent” cell is capable of giving rise to at least one cell type from each of two or more different organs or tissues of an organism, wherein the said cell types may originate from the same or from different germ layers, but is not capable of giving rise to all cell types of an organism. A “unipotent” cell is capable of differentiating to cells of only one cell lineage.

The term “mesenchymal stem cells” is to be understood as multipotent stromal cells derived or isolated from principally mesenchymal or from stromal cells. Said mesenchymal stem cells are able to differentiate various cell types, including but not limiting to hepatocytes, osteoblasts, chondrocytes, tenocytes and adipocytes.

The term “hepatocyte” encompasses epithelial and parenchymal liver cells, including but not limited to hepatocytes of different sizes or ploidy (e.g., diploid, tetraploid, octaploid).

The term “HLA-E positive” as used herein, refers to liver progenitor or stem cells that expresses a detectable level of HLA-E protein. HLA-E expression may be intracellular, on the cell surface and/or as soluble protein. When a cell is said to be positive for a particular marker, this means that a skilled person will conclude the presence or evidence of a distinct signal, e.g., antibody-detectable or detection possible by reverse transcription polymerase chain reaction or flow cytometry, for that marker when carrying out the appropriate measurement, compared to suitable controls. Where the detection method allows for quantitative assessment of the marker, positive cells may on average generate a signal that is significantly different from the control, e.g., but without limitation, at least 0.5 fold, at least 1 fold, at least 1.5-fold higher than such signal generated by control cells, e.g., at least 2-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold higher or even higher.

The term “elevated” when relating to elevated expression, secretion or enzymatic activity, as used herein, defines a level of expression, secretion or enzymatic activity as the result of preconditioning which is higher than the basal level of expression, secretion or enzymatic activity in similar cells that have not been submitted to any treatment such as for instance preconditioning, for example, but without limitation, at least 0.5 fold, at least 1 fold, at least at least 1.5-fold higher than such signal generated by control cells, e.g., at least 2-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold higher or even higher.

The term “liver” refers to liver organ. The term “part of liver” generally refers to a tissue sample derived from any part of the liver organ, without any limitation as to the quantity of the said part or the region of the liver organ where it originates. Preferably, all cell types present in the liver organ may also be represented in the said part of liver. Quantity of the part of liver may at least in part follow from practical considerations to the need to obtain enough primary liver cells for reasonably practicing the method of the invention. Hence, a part of liver may represent a percentage of the liver organ (e.g. at least 1%, 10%, 20%, 50%, 70%, 90% or more, typically w/w). In other non-limiting examples, a part of liver may be defined by weight (e.g. at least 1 g, 10 g, 100 g, 250 g, 500 g, or more). For example, a part of liver may be a liver lobe, e.g., the right lobe or left lobe, or any segment or tissue sample comprising a large number of cells that is resected during split liver operation or in a liver biopsy.

The term “adult liver” refers to liver of subjects that are post-natal, i.e. any time after birth, preferably full term, and may be, e.g., at least at least 1 day, 1 week, 1 month or more than 1 month of age after birth, or at least 1, 5, 10 years or more. Hence, an “adult liver”, or mature liver, may be found in human subjects who would otherwise be described in the conventional terms of “infant”, “child”, “adolescent”, or “adult”. A skilled person will appreciate that the liver may attain substantial developmental maturity in different time postnatal intervals in different animal species, and can properly construe the term “adult liver” with reference to each species.

The term “mammal” includes any animal classified as such, including, but not limited to, humans, domestic and farm animals, zoo animals, sport animals, pet animals, companion animals and experimental animals, such as, for example, mice, rats, rabbits, dogs, cats, cows, horses, pigs and primates, e.g., monkeys and apes.

The term “disassociating” as used herein generally refers to partly or completely disrupting the cellular organization of a tissue or organ, i.e., partly or completely disrupting the association between cells and cellular components of a tissue or organ. As can be understood by a skilled person, the aim of disassociating a tissue or organ is to obtain a suspension of cells (a cell population) from the said tissue or organ. The suspension may comprise solitary or single cells, as well as cells physically attached to form clusters or clumps of two or more cells. Disassociating preferably does not cause or, causes as small as possible reduction in cell viability.

As used herein, the term “primary cell” includes cells present in a suspension of cells obtained from a tissue or organ of a subject, e.g. liver, by disassociating cells present in such explanted tissue or organ with appropriate techniques.

The term “culturing” is common in the art and broadly refers to maintenance and/or growth of cells and/or progeny thereof.

The term “passaging” is common in the art and refers to detaching and dissociating the cultured cells from the culture substrate and from each other. For sake of simplicity, the passage performed after the first time of growing the cells under adherent culture conditions is herein referred to as “first passage” (or passage 1, P1) within the method of the invention. The cells may be passaged at least one time and preferably two or more times. Each passage subsequent to passage 1 is referred to herein with a number increasing by 1, e.g., passage 2, 3, 4, 5, or P1, P2, P3, P4, P5, etc.

The term “confluence” as used herein refers to a density of cultured cells in which the cells contact one another covering substantially all of the surfaces available for growth (i.e., fully confluent).

The term “plasma” is as conventionally defined and refers to a composition which does not form part of a human or animal body.

The term “serum”, as conventionally defined, is obtained from a sample of whole blood by first allowing clotting to take place in the sample and subsequently separating the so formed clot and cellular components of the blood sample from the liquid component (serum) by an appropriate technique, typically by centrifugation. An inert catalyst, e.g., glass beads or powder, can facilitate clotting. Advantageously, serum can be prepared using serum-separating vessels (SST), which contain the inert catalyst to mammals.

The term “cell medium” or “cell culture medium” or “medium” refers to an aqueous liquid or gelatinous substance comprising nutrients which can be used for maintenance or growth of cells. Cell culture media can contain serum or be serum-free.

The term “growth factor” as used herein refers to a biologically active substance which influences proliferation, growth, differentiation, survival and/or migration of various cell types, and may effect developmental, morphological and functional changes in an organism, either alone or when modulated by other substances. A growth factor may typically act by binding, as a ligand, to a receptor (e.g., surface or intracellular receptor) present in cells. A growth factor herein may be particularly a proteinaceous entity comprising one or more polypeptide chains. The term “growth factor” encompasses the members of the fibroblast growth factor (FGF) family, bone morphogenic protein (BMP) family, platelet derived growth factor (PDGF) family, transforming growth factor beta (TGF-beta) family, nerve growth factor (NGF) family, the epidermal growth factor (EGF) family, the insulin related growth factor (IGF) family, the hepatocyte growth factor (HGF) family, the interleukin-6 (IL-6) family (e.g. oncostatin M), hematopoietic growth factors (HeGFs), the platelet-derived endothelial cell growth factor (PD-ECGF), angiopoietin, vascular endothelial growth factor (VEGF) family, or glucocorticoids. Where the method is used for human liver cells, the growth factor used in the present method may be a human or recombinant growth factor. The use of human and recombinant growth factors in the present method is preferred since such growth factors are expected to elicit a desirable effect on cellular function.

As used herein, the term “preconditioned” liver progenitor cell or stem cell is defined as a liver progenitor cell or stem cell exposed in vitro to one or more signaling molecules. By preference, said molecules are added to the cell medium of said cells, for a predefined period of time.

The term “cytokine” as used herein, refers to a signaling molecule such as a growth, differentiation or chemotrophic factor secreted by immune or other cells, whose action is on cells of the immune system or on multipotent cells, such as, but not limited to, T-cells, B-cells, NK cells, macrophages, multipotent cells including hematopoietic cells, mesenchymal stem cells and progenitor cells, or other cell types. Representative cytokines include, but are not limited to, indoleamine 2,3-dioxygenase (IDO), hepatocyte growth factor (HGF), the group consisting of interleukins such as but not limited to interleukin-1 alpha (IL-1α), interleukin-1 beta (IL-1β), interleukin-2 (IL-2), interleukin-3 (IL-3), interleukin-4 (IL-4), interleukin-6 (IL-6), interleukin-10 (IL-10), interferons such as interferon-alpha (IFNα) and interferon-gamma (IFNγ), tumor necrosis factor-alpha (TNFα), and granulocyte-macrophage colony stimulating factor.

The terms “cell population” and “population of cells” refer generally to a group of cells. Unless indicated otherwise, the term refers to a cell group consisting essentially of or comprising cells as defined herein. A cell population may consist essentially of cells having a common phenotype or may comprise at least a fraction of cells having a common phenotype. Cells are said to have a common phenotype when they are substantially similar or identical in one or more demonstrable characteristics, including but not limited to morphological appearance, the level of expression of particular cellular components or products (e.g., RNA or proteins), activity of certain biochemical pathways, proliferation capacity and/or kinetics, differentiation potential and/or response to differentiation signals or behavior during in vitro cultivation (e.g., adherence or monolayer growth). Such demonstrable characteristics may therefore define a cell population or a fraction thereof. A cell population may be “substantially homogeneous” if a substantial majority of cells have a common phenotype. A “substantially homogeneous” cell population may comprise at least 60%, e.g., at least 70%, at least 80%, at least 90%, at least 95%, or even at least 99% of cells having a common phenotype, such as the phenotype specifically referred to (e.g., the phenotype of liver progenitor or stem cells of the invention, or to progeny of liver progenitor or stem cells of the invention). Moreover, a cell population may consist essentially of cells having a common phenotype such as the phenotype of liver progenitor or stem cells of the invention (i.e. a progeny of liver progenitor or stem cells of the invention) if any other cells present in the population do not alter or have a material effect on the overall properties of the cell population and therefore it can be defined as a cell line.

The term “pharmaceutically acceptable carrier” as used herein, refers to a carrier or a diluent that does not cause significant irritation to a subject and does not abrogate the biological activity and properties of the administered composition. Examples, without limitations, of carriers are propylene glycol, saline, emulsions and mixtures of organic solvents with water.

The term “sufficient amount” means an amount sufficient to produce a desired and measurable effect, e.g., an amount sufficient to alter a protein expression profile.

The term “therapeutically effective amount” is an amount that is effective to ameliorate a symptom of a disease. A therapeutically effective amount can be a “prophylactically effective amount” as prophylaxis can be considered therapy.

The term “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

The term “allogeneic” as used herein means that the donated material comes from different individual than the recipient. Allogeneic stem cell transplantation refers to a procedure in which a person receives stem cells from a genetically similar, but not identical, donor.

The term “fibrosis” as used herein refers to the formation of excess fibrous connective tissue in an organ or tissue in a reparative or reactive process.

The term “liver fibrosis” refers to the accumulation of interstitial or “scar” extracellular matrix after either acute or chronic liver injury. Cirrhosis, the end-stage of progressive fibrosis, is characterized by septum formation and rings of scar that surround nodules of hepatocytes. Typically, fibrosis requires years or decades to become clinically apparent, but notable exceptions in which cirrhosis develops over months may include pediatric liver disease (e.g. biliary atresia), drug-induced liver disease, and viral hepatitis associated with immunosuppression after liver transplantation.

The term “rMFI” or relative median fluorescence intensity is the ratio between the fluorescence intensity measured by use of an antibody to a specific target versus the intensity obtained from a control antibody (isotype control).

The term “genetic engineering”, “genetic modification” or “genetic manipulation”, is the direct manipulation of an organism's genes using biotechnology. It is a set of technologies used to change the genetic makeup of cells, including the transfer of genes within and across species boundaries to produce improved or novel organisms. New DNA is obtained by either isolating and copying the genetic material of interest using recombinant DNA methods or by artificially synthesizing the DNA. A construct is usually created and used to insert this DNA into the host organism.

The term “genome editing” or “genome engineering” is a type of genetic engineering in which DNA is inserted, deleted, modified or replaced in the genome of a living organism. Unlike early genetic engineering techniques that randomly inserts genetic material into a host genome, genome editing targets the insertions to site specific locations.

In a first aspect, the invention provides compositions of human adult liver-derived progenitor or stem cells wherein at least 60% of said progenitor or stem cells express cell surface marker HLA-E or an isolated liver progenitor cell or stem cell which is HLA-E positive.

The induction of HLA-E expression in an isolated liver-derived progenitor cell is shown to considerably enhance its immunosuppressive properties. Said HLA-E expressing cells were protected against CD8+ T-cells mediated cytolysis. Without wishing to be bound by theory, the immunosuppressive properties of HLA-E positive progenitor cells are thought to be caused by the ability of HLA-E to regulate the cytotoxic activity of natural killer cells. Consequently, the HLA-E positive progenitor cells are particularly well suited for treatment and/or prevention of disorders involving unwanted immune responses, for example, but not limited to inflammation, auto-immune disease and graft rejection, including multiple liver disorders.

It is believed that these progenitor cells are able to retain their proliferation capacity and incubation in specific media allows the cells to differentiate specifically into liver-specific cell types. Their proliferation capacity, and their liver specificity lead to an increased efficacy and safety for liver cell transplantation. Furthermore, because of their adult origin, these stem cells obviate the immunological, ethical and carcinogenic issues associated to embryonic cells.

The enhanced immunosuppressive character of the progenitor cells of the invention contributes to a decrease the immune-response related risks of cell therapy and/or tissue transplant, which are particularly high during allogeneic cell transplantations. Accordingly, the HLA-E positive liver-derived progenitor cells of the invention are more likely to be successful as a cell therapy for disorders with unwanted immune responses, for example, but not limited to inflammation, auto-immune disease and graft rejection, as well as for multiple liver disorders, and with improved efficacy and tolerability for the patient.

In a particular embodiment, the invention provides a human liver progenitor cell which is HLA-E positive, preferably a human adult liver progenitor cell which is HLA-E positive.

For the purpose of the current invention, the terms “expression of HLA-E” or “HLA-E positive cells” are defined as an expression that is higher than the basal HLA-E expression that may be observed in wild-type, primary non-treated cells. Accordingly, said cells have elevated levels of HLA-E expression as compared to a basal HLA-E expression in non-treated progenitor or stem cells. Hence, the invention equally provides a composition of progenitor or stem cells isolated from liver which have enhanced levels of HLA-E expression compared to the basal level. The basal HLA-E expression can relate to the absence of expression as well as to a minimum of expression in the absence of a stimulus that enhances the HLA-E expression. An elevated level of expression refers to for example, but without limitation, a level of expression which is measured at least 1.5-fold higher than the measurement of expression by control cells, e.g., at least 2-fold, at least 4-fold, at least 10-fold, at least 20-fold, at least 30-fold, at least 40-fold, at least 50-fold higher or even higher.

In an embodiment, the current invention relates to a composition of human adult liver-derived progenitor or stem cells wherein at least 65%, more preferably at least 70%, more preferably at least 75%, more preferably at least 80%, more preferably at least 85%, more preferably at least 90%, more preferably at least 95% of said progenitor or stem cells express cell surface marker HLA-E.

In another or further embodiment, the current invention relates to compositions or liver progenitor or stem cells wherein the HLA-E expressing cells have an HLA-E relative median fluorescence intensity (rMFI) of at least 6.5 as measured by flow cytometry. The rMFI is the ratio obtained by dividing the median fluorescence intensity of the target by the median fluorescence intensity of the control. The rMFI is generally considered a stable parameter which is not subjected to variation. In the context of the current invention, an anti-(human) HLA-E antibody is used. An example of a suitable antibody is anti-HLA-E antibody clone REA1031 from Miltenyi. Possible flow cytometer is the MACSQuant® analyzer.

In a further embodiment, the HLA-E expressing cells exhibit an HLA-E relative medium fluorescence intensity (rMFI) of at least 7, more preferably at least 8, more preferably at least 9, more preferably at least 10 as measured by flow cytometry. In another embodiment, said rMFI is between 6.5 and 15, more preferably between 6.5 and 14, more preferably between 6.5 and 13, more preferably between 6.5 and 13, more preferably between 6.5 and 12.

Another protein involved in the induction of immune tolerance is HLA-G. Like HLA-E, HLA-G can also trigger the inhibitory natural killer cell receptor and exert an immunosuppressive function. HLA-E and HLA-G have been reported to work together. Accordingly, and in a further embodiment of the invention, the liver progenitor cells are further positive for HLA-G. The co-operation of HLA-E and HLA-G further enhances the immunosuppressive action of the respective liver progenitor cells, making them better suited for use in cell therapy approaches.

In an embodiment, the cell population or composition according to the current invention will show a higher HLA-G secretion.

In a further embodiment, cells of the invention, populations and compositions comprising said cells may be positive for at least one mesenchymal marker Mesenchymal markers include but are not limited to Vimentin, CD13, CD90, CD73, CD44, CD29, α-smooth muscle actin (ASMA) and CD140b. In a further embodiment, cells of the invention, populations and compositions comprising said cells secrete HGF. In another further embodiment, cells of the invention, populations and compositions comprising said cells are negative for HLA-DR. In a further embodiment, said populations and compositions are further optionally positive for at least one hepatic marker and/or a marker linked to at least one liver-specific activity. For example, hepatic markers include but are not limited to albumin (ALB), HNF-3B, HNF-4, CYP1A2, CYP2C9, CYP2E1, CYP3A4 and alpha-1 antitrypsin. Liver-specific activities include but are not limited to urea secretion, bilirubin conjugation, alpha-1-antitrypsin secretion, CYP3A4 activity. The presence of these markers/activities further assure the liver origin, the multipotency and the maintained liver functionality of the isolated liver progenitor cells or stem cells.

In an embodiment, the cells may also be characterized in terms of being
a. positive for α-smooth muscle actin (ASMA), CD140b and optionally albumin (ALB);
b. negative for Sushi domain containing protein 2 (SUSD2) and Cytokeratin-19 (CK-19).

In a further embodiment, said cells were confirmed to express CD90, CD73, Vimentin, ASMA, CD140b and CD13, and to secrete HGF.

In a further or other embodiment, the cells may also be characterized or measured as positive for
a. at least one hepatic marker selected from HNF-3B, HNF-4, CYP1A2, CYP2C9, CYP2E1 and CYP3A4 and optionally albumin; and/or
b. at least one mesenchymal marker selected from Vimentin, CD90, CD73, CD44, and CD29; and/or
c. at least one liver-specific activity selected from urea secretion, bilirubin conjugation, alpha-1-antitrypsin secretion, and CYP3A4 activity; and/or
d. at least one marker selected from ATP2B4, ITGA3, TFRC, SLC3A2, CD59, ITGB5, CD151, ICAM1, AN PEP, CD46, and CD81; and/or
e. at least one marker selected from MMPI, ITGA11, FMOD, KCND2, CCL11, ASPN, KCNK2, and HMCN1.

In a further embodiment, the cells are further tested and established as being negative for the markers CD133, CD45, CK19 and/or CD31.

In a further embodiment of the invention, the liver-derived progenitor cells further have elevated levels of secretion of prostaglandin E2 (PGE2) as compared to the basal level of PGE2 secretion detected in a non-treated liver-derived progenitor or stem cell.

In another further embodiment of the invention, the liver-derived progenitor cells further have elevated levels of indoleamine 2,3-dioxygenase (IDO) enzymatic activity as compared to the basal enzymatic activity detected in a non-treated liver progenitor or stem cell.

In yet another further embodiment of the invention, the liver-derived progenitor cells further have elevated levels of expression of one or more cytokines or immunomodulatory factors belonging to the group consisting of IDO, prostaglandin-endoperoxide synthase 2 (PTGS2), interleukin-6 (IL-6) and/or IL-10, as compared to the basal expression level detected in a non-treated liver progenitor or stem cell.

PTGS2 is involved in the production of PGE2 while IDO, PGE2, IL-6, IL-10 and HGF are secreted factors with immunoregulatory properties. Their individual or combined elevated levels of expression, secretion and/or enzymatic activity further contribute to the immunosuppressive and immunomodulatory properties of the liver-derived progenitor cells of the invention. Therefore, cells according to the abovementioned embodiments have stronger immunomodulatory properties and are safer for use in cell therapies, particularly in therapies involving allogeneic cell transplantation.

In an embodiment, said composition or cell population may comprise about 50% or more, about 60% or more, about 65% or more, about 70% or more, about 80% or more or about 90% or more of the progenitor or stem cells or is a substantially homogeneous or homogeneous population of the said progenitor or stem cells. The immunosuppressive properties of the composition or cell population are further enhanced by the increasing amount of progenitor or stem cells within said population.

In one embodiment of the invention the composition further comprises a pharmaceutically acceptable carrier. The pharmaceutically acceptable carrier or diluent is chosen wherein the cells of the invention remain viable and retain their immunomodulatory properties. The carrier can be a pharmaceutically acceptable solvent or dispersing medium containing, for example, water, saline, phosphate buffered saline, polyol (for example, glycerol, propylene glycol, liquid polyethylene glycol, and the like) and suitable mixtures thereof. The invention thus discloses a pharmaceutical composition comprising the isolated liver progenitor or stem cells as described above, cell lines thereof, or a cell population comprising such.

Preferably, a composition comprising the isolated liver progenitor cells of the invention may comprise at least 10³, 10⁶, 10⁹ or more cells (for example, between 5 million and 500 million or between 5 million and 250 million or between 50 million and 500 million or between 50 million and 250 million or between 100 million and 500 million or between 100 million and 250 million of cells for each dose or administration). Such cell-based compositions may also include other agents of biological (e.g. antibodies or growth factor) or chemical origin (e.g. drugs, cell preserving or labelling compounds) that may provide a further therapeutic, diagnostic, or any other useful effect. The literature provides several examples of optional additives, excipients, vehicles, and/or carrier that are compatible with cell-based pharmaceutical compositions that may include further specific buffers, growth factors, or adjuvants, wherein the amount of each component of the composition is defined (in terms of micrograms/milligrams, volume, or percentage), as well as the means to combine them with liver progenitor cells.

In a further embodiment compositions of the invention can be provided as pharmaceutical compositions that can be used in therapeutic methods for in vivo administration (in humans or in animal models) or in vitro applications in the form of a composition including such cells either as fresh cells or cells suitable for long-term storage (e.g. cryopreserved cells).

In another or further embodiment the composition of the invention can be provided as a suspension of cells, a sponge or other three-dimensional structure where cells can grow and differentiate in vitro and/or in vivo including bioartificial liver devices, natural or synthetic matrices, or other systems allowing the engraftment and functionality of cells in appropriate locations (including areas of inflammation or tissue injury that expressing chemokines that help the homing and the engraftment of the cells). In particular, the composition of the invention can be administered via injection (encompassing also catheter administration, intravenously or intra-arterially) or implantation, e.g. localized injection, systemic injection, intrasplenic injection, intra-articular injection, intraperitoneal injection, intraportal injection, injection to liver pulp, e.g., beneath the liver capsule, parenteral administration, or intrauterine injection into an embryo or fetus.

Cells, compositions and cell populations according to the current invention may be obtained via various methods.

In an embodiment, said stem or progenitor cells of the current invention and present in the composition or cell population comprise an exogenous sequence of nucleic acid encoding for HLA-E. The sequence of human HLA-E is known under Gene ID 3133 in NCBI. Introduction of said exogenous nucleic acid may be performed by genetic engineering techniques regularly known in the art. Said exogenous nucleic acid may be e.g., a nucleic acid construct comprising a vector, inducible promoter, a sequence encoding a protein product of interest, etc.). A variety of vectors (e.g. plasmids, expression vectors, retroviral vectors, etc.) are known in the art for the delivery of sequences into a cell. In an embodiment, the vector is a retroviral or lentiviral vector.

Various techniques known in the art may be used to transfect the target cells, e.g. electroporation, calcium precipitated DNA, fusion, transfection, lipofection and the like. The particular manner in which the DNA is introduced is not critical to the practice of the invention. Combinations of retroviruses and an appropriate packaging line may be used, where the capsid proteins will be functional for infecting the target cells. Usually, the cells and virus will be incubated for at least about 24 hours in the culture medium. Commonly used retroviral vectors are “defective”, i.e. unable to produce viral proteins required for productive infection. Replication of the vector requires growth in the packaging cell line.

Protein products of interest may also be selected to aid in detecting and/or selecting for progenitor or stem cell populations as described herein. For detecting or selecting stem cells, the detection construct is introduced into a cell or population of cells, suspected of being or comprising stem cells. After introduction of the expression construct, the cells are maintained for a period of time sufficient to express the detectable marker, usually at least about 12 hours and not more than about 2 weeks, and may be from about 1 day to about 1 week.

Genetic constructs may be removed from the target cells after expansion. This can be accomplished by the use of a transient vector system, or by including a heterologous recombination site that flanks the desired protein coding sequence. Preferably a detectable marker, e.g. green fluorescent protein, luciferase, cell surface proteins suitable for antibody selection methods, etc. is included in the expression vector, such that after deletion of the construct the cells can be readily isolated that lack the exogenous sequence.

Expression vectors that provide for the transient or long-term expression in mammalian cells may be used. In general, transient expression involves the use of an expression vector that is able to replicate efficiently in a host cell, such that the host cell accumulates many copies of the expression vector and, in turn, synthesizes high levels of a desired polypeptide encoded by the expression vector. Transient expression systems, comprising a suitable expression vector and a host cell, allow for the convenient short-term expansion of cells, but do not affect the long-term genotype of the cell.

In some embodiments, the selected cells are maintained in culture for at least one passage, usually at least about two passages; at least about three passages; or more, and not more than about 10 passages, usually not more than about seven passages. Following such culture, the cells are sorted for expression of the detectable marker as described above.

In an embodiment, said exogenous nucleic acid is introduced in said cells via targeted genome editing. Common methods used for targeted genome editing are the use of nucleases, such as meganucleases, zinc finger nucleases, transcription activator-like effector-based nucleases (TALEN), and the clustered regularly interspaced short palindromic repeats (CRISPR/Cas9) system. In a preferred embodiment, the CRISPR/Cas9 system is used to introduce HLA-E into said stem or progenitor cells.

The invention equally provides a method for preparing HLA-E positive liver progenitor cells or stem cells by means of pre-conditioning or stimulation. In particular, the method comprises the step of preconditioning of the cells, which refers to adding one or more cytokines to the cell medium of a culture of liver progenitor or stem cells.

In a preferred embodiment, such method comprises the steps of:
(a) Disassociating adult liver or a part thereof to form a population of primary liver cells;
(b) Generating preparations of the primary liver cells of (a);
(c) Culturing the cells comprised in the preparations of (b) onto a support which allows adherence and growth of cells thereto and the emergence of a population of cells;
(d) Passaging the cells of (c) at least once;
(e) At one passage of step (d), preferably at last passage of step (d), preconditioning the cells with one or more cytokines; and
(f) Harvesting the cell population that is obtained after preconditioning of (e).

In a further embodiment, the expression of HLA-E is optionally measured in the cells obtained in step (f) and if desired, further selection of these cells is performed.

The basal HLA-E expression can relate to the absence of expression as well as to a minimum of expression in the absence of exogenous cytokines in the medium.

Concerning Step (a) of the method, the dissociation step involves obtaining a liver or a part thereof that comprises, together with fully differentiated hepatocytes, an amount of primary cells that can be used for producing liver progenitor or stem cells.

The liver or part thereof is obtained from a “subject”, “donor subject” or “donor”, interchangeably referring to a vertebrate animal, preferably a mammal, more preferably a human. A part of a liver can be a tissue sample derived from any part of the liver and may comprise different cell types present in the liver. The cells according to the invention are preferably isolated from mammalian liver or part of a liver, where the term mammalian refers to any animal classified as a mammal, including humans, domestic and farm animals, and zoo, laboratory, sports, or pet animals, such as dogs, horses, cats, cows, mice, rats, rabbits, etc. More preferably, the liver progenitor cell or stem cell is isolated from human liver or a part thereof, preferably human adult liver or a part thereof. Liver progenitor cells or cell lines, or progeny thereof, derived according to the invention from livers of adult human subjects, can be advantageously used, e.g., in research and in therapy of patients, especially human patients, suffering from disorders involving unwanted immune responses, for example, but not limited to inflammation, auto-immune disease and graft rejection, including liver diseases. In contrast to other sources of stem cells, such as for example embryonic stem cells which are prone to generate tumor growth, stem cells derived from adult livers offer a reduction in the risk for carcinogenic deviation making them safer for use in cell transplantation.

In an alternative embodiment of the invention, the adult liver or part thereof may be from a non-human animal subject, preferably a non-human mammal subject. Progenitor or stem cells or cell lines, or progeny thereof, derived as described herein from livers of non-human animal or non-human mammal subjects can be advantageously used, e.g., in research and in the therapy of liver disease in members of the same, related or other non-human animal or non-human mammal species, or even in the therapy of human patients suffering from liver disease (e.g., xenotransplantation, bio-artificial liver devices comprising non-human animal or non-human mammal cells). By means of example and not limitation, particularly suitable non-human mammal cells for use in human therapy may originate from pigs.

A donor subject may be living or dead, as determined by art-accepted criteria, such as, for example, the “heart-lung” criteria (usually involving an irreversible cessation of circulatory and respiratory functions) or the “brain death” criteria (usually involving an irreversible cessation of all functions of the entire brain, including the brainstem). Harvesting may involve procedures known in the art, such as, for example, biopsy, resection or excision.

A skilled person will appreciate that at least some aspects of harvesting liver or part thereof from donor subjects may be subject to respective legal and ethical norms. By means of example and not limitation, harvesting of liver tissue from a living human donor may need to be compatible with sustenance of further life of the donor. Accordingly, only a part of liver may typically be removed from a living human donor, e.g., using biopsy or resection, such that an adequate level of physiological liver functions is maintained in the donor. On the other hand, harvesting of liver or part thereof from a non-human animal may, but need not be compatible with further survival of the non-human animal. For example, the non-human animal may be humanely culled after harvesting of the tissue. These and analogous considerations will be apparent to a skilled person and reflect legal and ethical standards.

Liver or part thereof may be obtained from a donor, preferably a human donor, who has sustained circulation, e.g., a beating heart, and sustained respiratory functions, e.g., breathing lungs or artificial ventilation. Subject to ethical and legal norms, the donor may need to be or need not be brain dead (e.g., removal of entire liver or portion thereof, which would not be compatible with further survival of a human donor, may be allowed in brain dead human beings). Harvesting of liver or part thereof from such donors is advantageous, since the tissue does not suffer substantial anoxia (lack of oxygenation), which usually results from ischemia (cessation of circulation).

Alternatively, liver or part thereof may be obtained from a donor, preferably a human donor, who at the time of harvesting the tissue has ceased circulation, e.g., has a non-beating heart, and/or has ceased respiratory functions, e.g., has non-breathing lungs and no artificial ventilation. While liver or part thereof from these donors may have suffered at least some degree of anoxia, viable progenitor or stem cells can also be isolated from such tissues. Liver or part thereof may be harvested within about 24 h after the donor's circulation (e.g., heart-beat) ceased, e.g., within about 20 h, e.g., within about 16 h, more preferably within about 12 h, e.g., within about 8 h, even more preferably within about 6 h, e.g., within about 5 h, within about 4 h or within about 3 h, yet more preferably within about 2 h, and most preferably within about 1 h, such as, within about 45, 30, or 15 minutes after the donor's circulation (e.g., heart-beat) ceased.

The harvested tissues may be cooled to about room temperature, or to a temperature lower than room temperature, but usually freezing of the tissue or parts thereof is avoided, especially where such freezing would result in nucleation or ice crystal growth. For example, the tissue may be kept at any temperature between about 1° C. and room temperature, between about 2° C. and room temperature, between about 3° C. and room temperature or between about 4° C. and room temperature, and may be advantageously be kept at about 4° C. The tissue may also be kept “on ice” as known in the art. The tissue may be cooled for all or part of the ischemic time, i.e., the time after cessation of circulation in the donor. That is, the tissue can be subjected to warm ischemia, cold ischemia, or a combination of warm and cold ischemia. The harvested tissue may be so kept for, e.g., up to 48 h before processing, preferably for less than 24 h, e.g., less than 16 h, more preferably for less than 12 h, e.g., less than 10 h, less than 6 h, less than 3 h, less than 2 h or less than 1 h.

The harvested tissue may advantageously be but need not be kept in, e.g., completely or at least partly submerged in, a suitable medium and/or may be but need not be perfused with the suitable medium, before further processing of the tissue. A skilled person is able to select a suitable medium which can support the survival of the cells of the tissue during the period before processing.

Isolation of progenitor cells or stem cells from a liver or part of a liver is performed according to methods known in the art, for example as described in EP1969118, EP3039123, EP3140393 or WO2017149059.

Briefly, a population of liver primary cells is first obtained from disassociating of liver or part thereof, to form a population of primary cells from said liver or part thereof. Subsequently, cells comprised in this preparation are cultured under adherent conditions, preferably as to allow adherence and growth of cells onto a support. Next, these cells are passaged at least once, preferably at 70% confluence. Finally, cells are isolated which are positive for at least one hepatic marker and at least one mesenchymal marker and that have at least one liver-specific activity.

A suitable method for disassociating liver or part thereof to obtain a population (suspension) of primary cells therefrom may be any method well known in the art, including but not limited to, enzymatic digestion, mechanical separation, filtration, centrifugation and combinations thereof.

Concerning step (b) of the method, the population of primary cells as defined and obtained herein by disassociating liver or part thereof may typically be heterogeneous, i.e., it may comprise cells belonging to one or more cell types belonging to any liver-constituting cell type, including progenitor or stem cells, that may have been present in liver parenchyma and/or in the liver non-parenchymal fraction. Exemplary liver-constituting cell types include but are not limited to hepatocytes, cholangiocytes (bile duct cells), Kupffer cells, hepatic stellate cells (Ito cells), oval cells and liver endothelial cells. The above terms have art-established meanings and are construed broadly herein as encompassing any cell type classified as such.

A primary cells population may comprise hepatocytes in different proportions (0.1%, 1%, 10%, or more of total cells), according to the method of disassociating liver and/or any methods for fractioning or enriching the initial preparation for hepatocytes and/or other cell types on the basis of physical properties (dimension, morphology), viability, cell culture conditions, or cell surface marker expression by applying any suitable techniques.

The population of primary cells as defined and obtained herein by disassociating liver (or part of it) can be used immediately for establishing cell cultures as fresh primary liver cells or, preferably, stored as cryopreserved preparations of primary liver cells using common technologies for their long-term preservation.

Concerning step (c), the preparation of liver primary cells obtained in step (b) is then cultured directly onto a fully synthetic support (e.g. plastic or any polymeric substance) or a synthetic support pre-coated with feeder cells, protein extracts, or any other material of biological origin that allow the adherence and the proliferation of similar primary cells and the emergence of a population of adult liver progenitor cells having the desired markers, such markers being identified preferably at the level of protein, by means of immunohistochemistry, flow cytometry, or other anti-body based technique. Primary cells are cultured in a cell culture medium sustaining their adherence and the proliferation of and the emergence of a homogenous cell population. This step of culturing of primary liver cells as defined above leads to emergence and proliferation of liver progenitor cells in the culture and can be continued until liver progenitor or stem cells have proliferated sufficiently. For example, culturing can be continued until the cell population has achieved a certain degree of confluence (e.g., at least 50%, 70%, or at least 90% or more confluent).

Liver progenitor or stem cells obtained at step (c) can be further characterized by technologies that allow detecting relevant markers already at this stage (that is, before passaging cells as indicated in step (d)), as described in EP3140393 or WO2017149059. Among the technologies used for identifying such markers and measuring them as being positive or negative, Western Blot, Flow Cytometry immunocytochemistry, and ELISA are preferred since these allow marker detection at the protein level even with the low amount of liver progenitor cells that are available at this step.

The isolation of a liver progenitor cell can then be made based on the presence of positive markers, the liver progenitor cells may be positive for at least one mesenchymal marker. Mesenchymal markers include but are not limited to Vimentin, CD13, CD90, CD73, CD44, CD29, a-smooth muscle actin (ASMA) and CD140-b. In addition, the liver progenitor cells may secrete HGF. In addition, the liver progenitor cells can optionally be positive for at least one hepatic marker and/or have at least one liver-specific activity. For example, hepatic markers include but are not limited to albumin (ALB), HNF-3B, HNF-4, CYP1A2, CYP2C9, CYP2E1, CYP3A4 and alpha-1 antitrypsin. Liver-specific activities include but are not limited to urea secretion, bilirubin conjugation, alpha-1-antitrypsin secretion and CYP3A4 activity.

Concerning Step (d) of the method, primary cells are cultured in a cell culture medium sustaining their adherence and the proliferation of and the emergence of a homogenous cell population that, following at least one passage, is progressively enriched for liver progenitor cells or stem cells. These liver progenitor cells can be rapidly expanded for generating sufficient cells for obtaining progeny having the desired properties (as described in EP3140393 or WO2017149059), with cell doubling that can be obtained within 48-72 hours and maintenance of liver progenitor cells having the desired properties for at least for 2, 3, 4, 5 or more passages.

The isolated liver progenitor cells are plated onto a substrate which allows adherence of cells thereto, and cultured in a medium sustaining their further proliferation, generally a liquid culture medium, which may contain serum or may be serum-free. In general, a substrate which allows adherence of cells thereto may be any substantially hydrophilic substrate. Current standard practice for growing adherent cells may involve the use of defined chemical media with or without addition of bovine, human or other animal serum. These media, that can be supplemented with appropriate mixture of organic or inorganic compounds may, besides providing nutrients and/or growth promoters, also promote the growth/adherence or the elimination/detachment of specific cell types. The added serum, besides providing nutrients and/or growth promoters, may also promote cell adhesion by coating the treated plastic surfaces with a layer of matrix to which cells can better adhere. As appreciated by those skilled in the art, the cells may be counted in order to facilitate subsequent plating of the cells at a desired density.

The environment in which the cells are plated may comprise at least a cell medium, in the methods of the invention typically a liquid medium, which supports the survival and/or growth of the isolated liver progenitor cells. The liquid culture medium may be added to the system before, together with or after the introduction of the cells thereto.

Typically, the medium will comprise a basal medium formulation as known in the art. Many basal media formulations can be used to culture the primary cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, Medium 199, Waymouth's MB 752/1 or Williams Medium E, and modifications and/or combinations thereof. Compositions of the above basal media are generally known in the art and it is within the skill of one in the art to modify or modulate concentrations of media and/or media supplements as necessary for the cells cultured. A preferred basal medium formulation may be one of those available commercially such as Williams Medium E, IMDM or DMEM, which are reported to sustain in vitro culture of adult liver cells, and including a mixture of growth factors for their appropriate growth, proliferation, maintenance of desired markers and/or biological activity, or long-term storage. Another preferred medium is commercially available serum-free medium that supports the growth of liver progenitor cells, such as e.g. StemMacs™ from Miltenyi.

Such basal media formulations contain ingredients necessary for mammal cell development, which are known per se. By means of illustration and not limitation, these ingredients may include inorganic salts (in particular salts containing Na, K, Mg, Ca, Cl, P and possibly Cu, Fe, Se and Zn), physiological buffers (e.g., HEPES, bicarbonate), nucleotides, nucleosides and/or nucleic acid bases, ribose, deoxyribose, amino acids, vitamins, antioxidants (e.g., glutathione) and sources of carbon (e.g. glucose, pyruvate, e.g., sodium pyruvate, acetate, e.g., sodium acetate), etc. It will also be apparent that many media are available as low-glucose formulations with or without sodium pyruvate.

For use in culture, basal media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., β-mercaptoethanol. While many basal media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin.

Hormones can also be advantageously used in cell culture and include, but are not limited to D-aldosterone, diethylstilbestrol (DES), dexamethasone, estradiol, hydrocortisone, insulin, prolactin, progesterone, somatostatin/human growth hormone (HGH), thyrotropin, thyroxine, L-thyronine, epithelial growth factor (EGF) and hepatocyte growth factor (HGF). Liver cells can also benefit from culturing with triiodothyronine, α-tocopherol acetate, and glucagon.

Lipids and lipid carriers can also be used to supplement cell culture media. Such lipids and carriers can include, but are not limited to cyclodextrin, cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others. Albumin can similarly be used in fatty-acid free formulations.

Also contemplated is supplementation of cell culture medium with mammalian plasma or sera. Plasma or sera often contain cellular factors and components that are necessary for viability and expansion. The use of suitable serum replacements is also contemplated. Suitable sera or plasmas for use in the media as described herein may include human serum or plasma, or serum or plasma from non-human animals, preferably non-human mammals, such as, e.g., non-human primates (e.g., lemurs, monkeys, apes), fetal or adult bovine, horse, porcine, lamb, goat, dog, rabbit, mouse or rat serum or plasma, etc. In another embodiment, the any combination of the above plasmas and/or sera may be used in the cell medium.

In a further embodiment of the invention, the isolated liver progenitor cells are preconditioned with one or more cytokines. Preferably, said cytokines are added to the cell medium of a culture of liver progenitor or stem cells. The cells which are employed may be fresh, frozen, or have been subject to prior culture.

Concerning step (e), the medium composition for preconditioning may have the same features, may be the same or substantially the same as the composition of medium used during the isolation of the liver progenitor cells. Alternatively, the medium may be different.

Preconditioning of isolated liver progenitor cells according to an embodiment of the invention thus involves exposing the cells in vitro to one or more signaling molecules, in this case cytokines, through addition of the respective cytokines to the cell medium. The cytokines suitable for use in preconditioning can be pro-inflammatory cytokines as well as anti-inflammatory cytokines and include but are not limited to interleukin-1 alpha (IL-1α), interleukin-1 beta (IL-1β), interleukin-2 (IL-2), interleukin-3 (IL-3), interleukin-4 (IL-4), interleukin-6 (IL-6), interleukin-10 (IL-10), interferons such as interferon-alpha (INFα) and interferon-gamma (IFNγ), tumor necrosis factor-alpha (TNFα) and granulocyte-macrophage colony stimulating factor.

The expression of specific markers such as HLA-E can be detected using any suitable immunological technique known in the art, such as flow cytometry, immuno-cytochemistry or affinity adsorption, Western blot analysis, ELISA, etc., or by any suitable technique of measuring the quantity of the marker mRNA, e.g., Northern blot, semi-quantitative or quantitative RT-PCR, etc.

As described above, in vitro treatment of isolated progenitor cells with cytokines leads to the induction of HLA-E expression. Accordingly, and in a further embodiment of the invention, the preconditioned cells have elevated levels of HLA-E expression as compared to a basal HLA-E expression in non-preconditioned liver progenitor or stem cells. Hence, the invention equally provides a method for preparing preconditioned progenitor cells isolated from liver which have enhanced levels of HLA-E expression compared to the basal level, by adding one or more cytokines to the cell medium. The basal HLA-E expression can relate to the absence of expression as well as to a minimum of expression in the absence of exogenous cytokines in the medium. An elevated level of expression refers to for example, but without limitation, a level of expression which is measured at least 1.5-fold higher than the measurement of expression by control cells, e.g., at least 2-fold, at least 4-fold, at least 10-fold, at least 20-fold, at least 30-fold, at least 40-fold, at least 50-fold higher or even higher.

By preference, cytokines i.e. IFNγ, TNFα, IL-1β, and optionally IL-10 and combinations thereof are used for inducing HLA-E expression. Accordingly, in a further embodiment of the invention preconditioning of the adult liver progenitor cells or stem cells is preferably performed with cytokines selected from IFNγ, TNFα, IL-1β, IL-10 and any combinations thereof. These cytokines were found to efficiently induce the expression of HLA-E in liver progenitor cells. In an embodiment the cytokines are at least IFNγ and TNFα. In a preferred embodiment, the mixture further comprises IL-1β and/or IL-10. These specific cytokine combinations show increasing efficiency in the induction of HLA-E expression in liver progenitor or stem cells.

In an embodiment the cytokines are added to the cell medium of the cells at a total final concentration of between 5 ng/ml and 400 ng/ml, preferably between 10 ng/ml and 360 ng/ml, more preferably between 20 ng/ml and 240 ng/ml. In another or further embodiment of the invention the individual cytokine concentration in the cell medium is between 5 and 100 ng/ml, more preferably between 10 and 80 ng/ml, most preferably between 20 and 60 ng/ml.

More in particular, the effectiveness of the preconditioning of the liver progenitor cells with cytokines is most effective on HLA-E expression when the cells are preconditioned for 1 hour to 72 hours, more preferably for 12 hours to 60 hours, more preferably for 24 hours to 60 hours, and most preferably for 24-48 hours.

The referred concentration and treatment duration ranges are deemed to be sufficient for inducing an acceptable HLA-E expression in said cells.

Concerning step (f) above, the optional isolation of a cell population in a further embodiment of the invention applies to cells that have an elevated level of HLA-E compared to non-preconditioned liver progenitor or stem cells.

As mentioned, the invention also relates to a composition comprising the isolated liver progenitor cells or stem cells which are positive for HLA-E of the invention, cell lines thereof or a cell population comprising such, or progeny thereof including differentiated progeny, especially hepatocytes or hepatocyte-like cells, optionally genetically modified.

In a further aspect the invention provides a composition as described above for use in the treatment of liver diseases, including but not limited to:
(allogenic) liver cell transplantation in order to treat liver metabolic deficiencies, liver degenerative diseases or fulminant liver failure,
the preparation of bio-artificial liver devices,
the preparation of animal models of human liver diseases thanks to transplantation of isolated progenitor or stem cell or population thereof according to the invention in animals,
the preparation of in vitro and animal models of toxicology, pharmacology,
testing new drugs on the isolated progenitor or stem cell or population thereof according to the invention, including antiviral drugs for human hepatitis viruses.

The present cells can therefore be used in the treatment of liver associated diseases including but not limited to liver failure, hepatitis and inborn errors of metabolism. The composition of the invention is especially well suited for use in the treatment of liver diseases associated with inflammation or immune responses owing to their immunomodulatory properties. Treatments involving liver cell transplantation, which encompass possible immune-related risks also benefit from the outstanding immunoregulatory properties of compositions comprising the liver progenitor cells of the invention.

The present invention also discloses the isolated progenitor or stem cell, population thereof or composition comprising the latter for use in the following purposes:
to treat disorders with unwanted immune response, for example, but not limited to inflammation, auto-immune diseases and graft rejection,
in immunotherapy,
to treat fibrotic disorders including but not limited to liver fibrotic disorders, pulmonary fibrosis, kidney fibrosis, prostate fibrosis, breast fibrosis, heart muscle fibrosis and other disorders involving the increase specific markers of fibrosis, that is any biochemical, serological markers or any other clinical or echographic characteristics, that can be correlated with the presence of fibrotic disease, liver fibrosis and/or chronic fibro-inflammatory diseases including fibro-inflammatory chronic liver failure,
liver progenitor or stem cell transplantation in order to treat liver based inborn, metabolic deficiencies:
Non exhaustive examples of such diseases include phenylketonuria and other aminoacidopathies, haemophilia and other clotting factor deficiencies, familial hypercholesterolemia and other lipid metabolism disorders, urea cycle disorders, glycogenosis, galactosemia, fructosemia, tyrosinemia, protein and carbohydrate metabolism deficiencies, organic aciduria, mitochondrial diseases, peroxisomal and lysosomal disorders, protein synthesis abnormalities, defects of liver cell transporters, defect of glycosylation and the like,
to treat acquired progressive liver degenerative diseases,
to treat fulminant liver failure and acute or chronic liver failure,
in bio-artificial liver devices and liver assist devices,
animal models of human liver diseases

The HLA-E positive liver progenitor cells of the invention or cell population thereof or composition containing them can be used for tissue engineering and cell therapy via liver cell transplantation (LCT) in intra-hepatic or extra-hepatic locations (including for modulating immunological response to the prior or subsequent transplantation of liver or other organs and tissues). Using this approach, animal models of human liver diseases can be also obtained by transplanting isolated human liver progenitor cells of the invention or a composition containing them in animals wherein the effects of a compound on human hepatocytes can be more effectively evaluated and distinguished from effects in the animal model.
in the preparation of animal models of human hepatotropic virus infections (HBV, HAV, HCV, HEV, HDV, . . . )
to study natural history, transmission, resistance, effects of treatment, use of antiviral drugs or any research using the transplanted liver progenitor or stem cell according to the invention,
the preparation of in vitro cell models and in vivo animal models of toxicology, pharmacology and pharmacogenetics using the liver progenitor or stem cell according to the invention,
the testing of new drugs on the liver progenitor or stem cell according to the invention,
gene therapy, by inserting viral sequences in the liver progenitor or stem cell according to the invention which can then be expanded in vitro,
animal models to study human liver cell metabolism,
tolerance of allogeneic cells
use of the liver progenitor or stem cell according to the invention to avoid, prevent or treat liver or liver cell allograft rejection.

As described above, due to the immunosuppressive properties of the liver progenitor cells of the invention, the composition of the invention can be administered to a tissue of interest to protect this tissue against inflammation, against fibrosis and against subsequent destruction. This is especially advantageous during cell replacement therapies where unwanted immune reactions often have detrimental consequences. The composition of the invention can thus be used in cell replacement therapies. The progenitor cells can be administered to a tissue of interest, preferably liver tissue, in a subject to supplement functioning cells or replace cells, which have lost function and where inflammation leading to fibrosis and tissue destruction is to be avoided. Accordingly, the progenitor cells, cell lines thereof, cell populations thereof and composition comprising the latter are especially well suited for use in the treatment of fibrotic disorders including but not limited to liver fibrotic disorders, pulmonary fibrosis, kidney fibrosis, prostate fibrosis, breast fibrosis, heart muscle fibrosis and other disorders involving the increase specific markers of fibrosis, that is any biochemical, serological markers or any other clinical or echographic characteristics, that can be correlated with the presence of fibrotic disease, in particular fibro-inflammatory chronic liver diseases where fibro-inflammatory reactions in the liver often result in chronic liver failure. The immunosuppressive properties of the liver-derived progenitor cells contribute to reduce or prevent ongoing fibro-inflammatory reactions that cause progressive loss of liver function. On the other hand, the ability of the liver progenitor cells to regenerate and to differentiate into liver cell types contribute to liver regeneration and thus help prevent chronic liver failure.

Disease states or deficiencies typified by liver fibrosis leading to loss of liver mass and/or function, and that could benefit from liver progenitor or stem cells having elevated levels of HLA-G of the invention include but are not limited to Alagille syndrome, alcoholic liver disease (alcohol-induced cirrhosis), alpha1-antitrypsin deficiency (all phenotypes), hyperlipidemias and other lipid metabolism disorders, autoimmune hepatitis, Budd-Chiari syndrome, biliary atresia, progressive familial cholestasis type I, II and III, cancer of the liver, Caroli Disease, Crigler-Najjar syndrome, fructosemia, galactosemia, carbohydrate deficient glycosylation defects, other carbohydrate metabolism disorders, Refsum disease and other peroxisomal diseases, Niemann Pick disease, Wolman disease and other lysosomal disorders, tyrosinemia, triple H, and other amino acid metabolic disorders, Dubin-Johnson syndrome, fatty liver diseases including Nonalcoholic fatty liver disease (NAFLD) and Nonalcoholic steatohepatitis (NASH), chronic liver failure, Acute-on-Chronic Liver Failure (ACLF), Gilbert Syndrome, Glycogen Storage Disease I and III, hemochromatosis, hepatitis A-G, porphyria, primary biliary cirrhosis, sclerosing cholangitis, tyrosinemia, clotting factor deficiencies, hemophilia B, phenylketonuria, Wilson's Disease, fulminant liver failure, post hepatectomy liver failure, mitochondrial respiratory chain diseases. In addition, the cells can also be used to treat acquired liver disorders due to viral infections.

Accordingly, the use of a progenitor cell or stem cell, cell line thereof, cell population and/or composition of the invention is provided for use in therapy and/or for use in the manufacture of a medicament for the treatment of liver diseases. Such diseases may include disorders affecting liver tissue, diseases affecting the hepatocyte viability and/or function as well as chronic liver failure caused by fibro-inflammatory reactions. In addition, owing to the immunomodulatory and immunosuppressive properties the use of a progenitor cell or stem cell, cell line thereof, cell population and/or composition of the invention is provided for use in therapy and/or for use in the manufacture of a medicament for the treatment of diseases wherein undesired immune responses are to be avoided. Administration of the cells according to the invention can lead to tissue reconstitution or regeneration in the subject. The cells are administered in a manner that permits them to graft or migrate to the intended tissue site and reconstitute or regenerate the functionally deficient area, or to protect that area against further damage possibly caused by inflammation reactions.

The present specification also describes a method for preventing and/or treating a liver disease, comprising administration of the composition of the invention to a subject in need of such treatment. Such administration is typically in therapeutically effective amount, i.e., generally an amount which provides a desired local or systemic effect and performance.

In a further aspect, the invention relates to a pharmaceutical composition comprising the adult liver progenitor or stem cells of the invention, cell lines thereof or a cell population comprising such. By means of example and not limitation, the isolated liver progenitor or stem cells of the invention, cell lines thereof or cell populations comprising such or progeny thereof can be advantageously administered via injection (encompassing also catheter administration) or implantation, e.g. localized injection, systemic injection, intrasplenic injection (see also Gupta et al., Seminars in Liver Disease 12: 321, 1992), injection to a portal vein, injection to liver pulp, e.g., beneath the liver capsule, parenteral administration, or intrauterine injection into an embryo or fetus.

The liver originated progenitor or stem cells of the invention, cell lines thereof or cell populations comprising such, or progeny thereof, optionally genetically modified, or a pharmaceutical composition comprising these, can further be used for tissue engineering and cell therapy via liver cell transplantation (LCT). Liver cell transplantation, and liver stem cell transplantation (LSCT) refers to the technique of infusing mature hepatocytes or liver progenitor cells, including the cells of the invention, in any way leading to hepatic access and engraftment of the cells, preferably via the portal vein, but also by direct hepatic injection, or by intrasplenic injection.

For example, the cells may be provided as a cell suspension in any preservation medium, preferably containing human albumin, after isolation procedure or after thawing following cryopreservation.

In an embodiment, the present invention contemplates using a patient's own liver tissue to isolate the progenitor or stem cells of the invention. Such cells would be autologous to the patient and could be readily administered to the patient. However, if the patient contained for example a genetic defect underlying a particular pathological condition, such defect could be averted by genetically manipulating the obtained cells or by using progenitor or stem cells isolated from tissue which is not the patient's own also known in the art as allogeneic stem cell transplantation.

Where administration of such cells to a patient is contemplated, it may be preferable that the liver tissue subjected to the method of the present invention to obtain the progenitor or stem cells, is selected such as to maximize, at least within achievable limits, the tissue compatibility between the patient and the administered cells. Despite these efforts, the risk of rejection of the administered cells by patient's immune system remains high (e.g., graft vs. host rejection). The composition comprising liver-derived progenitor cells according to the current invention further decreases the risk of rejection of allogeneic LCT or LSCT, therefore and in a further embodiment the invention provides a composition for use in allogeneic transplantation.

An issue concerning the therapeutic use of the progenitor or stem cells of the invention is the quantity of cells necessary to achieve an optimal effect. Doses for administration may be variable, may include an initial administration followed by subsequent administrations and can be ascertained by the skilled artisan armed with the present disclosure. Typically, the administered dose or doses will provide for a therapeutically effective amount of the cells, i.e., one achieving the desired local or systemic effect and performance. In addition, the skilled person can readily determine the optional additives, vehicles, and/or carrier in pharmaceutical compositions of the invention to be administered to a subject. Typically, any additives (in addition to the active progenitor or stem cell(s) and/or cytokine(s)) may be present in an amount of 0.001 to 50% (w/w or w/v) solution in phosphate buffered saline, and the active ingredient may be typically present in the order of micrograms to milligrams, such as about 0.0001 to about 5% (w/w or w/v), preferably about 0.0001 to about 1%, most preferably about 0.0001 to about 0.05% or about 0.001 to about 20%, preferably about 0.01 to about 10%, and most preferably about 0.05 to about 5%.

When administering a therapeutic composition comprising the HLA-E positive liver progenitor cells or population thereof, it may generally be formulated in a unit dosage. In any case, it may be desirable to include agents and/or adapt known methods for administering cells to patients that ensure viability of cells of the invention or population of cells thereof, for example by incorporating the cells into a biopolymer or synthetic polymer. Examples of suitable biopolymers include, but are not limited to, fibronectin, fibrin, fibrinogen, thrombin, collagen, and proteoglycans laminins, adhesion molecules, proteoglycans, hyaluronans, glycosaminoglycan chains, chitosan, alginate, natural or synthetically modified peptides that are derived from such proteins, and synthetic, biodegradable and biocompatible polymers. These compositions may be produced with or without including cytokines, growth factors, and administered as a suspension or as a three-dimensional gel with the cells embedded there within.

Sterile injectable solutions can be prepared by incorporating the cells utilized in practicing the present invention in the required amount of the appropriate solvent with various amounts of the other ingredients, as desired. Such compositions may further be in admixture with a suitable carrier, diluent, or excipient such as sterile water, physiological saline, glucose, dextrose, or the like. The compositions can contain auxiliary substances such as wetting or emulsifying agents, pH buffering agents, gelling or viscosity enhancing additives, preservatives, flavoring agents, colors, and the like, depending upon the route of administration and the preparation desired.

The invention is further described by the following non-limiting examples which further illustrate the invention, and are not intended to, nor should they be interpreted to, limit the scope of the invention.

### EXAMPLES

### 1. Preparation of Preconditioned HLA-E Positive Liver Progenitor or Stem Cells

### (a) Preparation of Liver Progenitor Cells

Human adult liver progenitor cells were isolated, as described in EP 3 140 393 or WO 2017 149 059, from livers of healthy cadaveric or non-heart beating donors.

Briefly, liver cell preparations are re-suspended in Williams' E medium supplemented with 10% FBS, 10 mg/ml INS, 1 mM DEX. The primary cells are cultured on Corning® CellBIND® flasks and cultured at 37° C. in a fully humidified atmosphere containing 5% CO₂. After 24 hours, medium is changed in order to eliminate the non-adherent cells and thereafter renewed twice a week, whereas the culture is microscopically followed every day. Culture medium is switched after 12-16 days to high glucose DMEM supplemented with 9% FBS. A cell type with mesenchymal-like morphology emerges and proliferates. When reaching 70-95% confluence, cells are trypsinized with recombinant trypsin and 1 mM EDTA and replated at a density of 1-10×10³ cells/cm². At each passage, cells were trypsinized at 80-90% confluency.

### (b) Preconditioning of Liver Progenitor Cells

Inflammation priming was carried out at P5 on cells reaching 80-90% confluence, with IL-1β (5-100 ng/mL, peprotech, ref: 200-1B), IFNγ (5-100 ng/mL, prospec, ref: CYT-206), TNFα (5-100 ng/mL, prospec, ref: CYT-223), IL-10 (5-100 ng/mL, peprotech, ref: 200-10-10UG) and/or combinations thereof for 48 h, wherein “All together” is used to refer to cells treated with a combination of IL-1β, IFNγ, TNFα and IL-10. Afterwards, supernatants were harvested and cells trypsinized with recombinant trypsin (trypLE; LifeTech) and 1 mM EDTA for analysis.

### 2. HLA-E Expression: Characterization of the Cells by Flow Cytometry

HLA-E expression on untreated and treated cells was evaluated by flow cytometry.

A total of 5×10⁵ cells were incubated in PBS buffer for 30 min at 4° C. or 15 min at room temperature with an anti-human HLA-E-PE antibody. Corresponding control isotype antibodies were used to evaluate non-specific binding of monoclonal antibodies. Shift of histograms from the isotype indicates increased expression of HLA-E compared to the isotype. These results show elevated levels of HLA-E expression in preconditioned cells compared to the untreated condition.

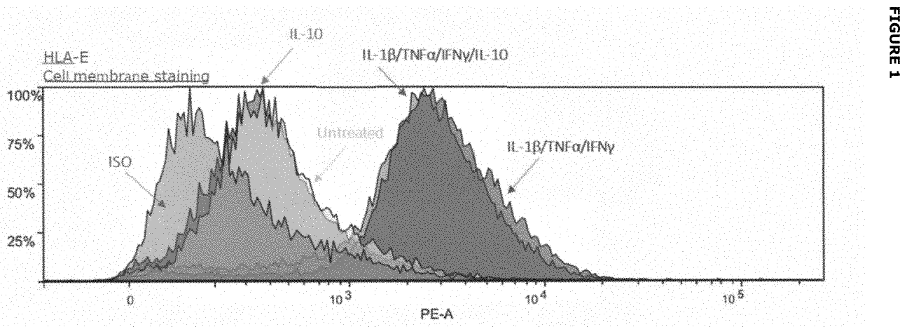

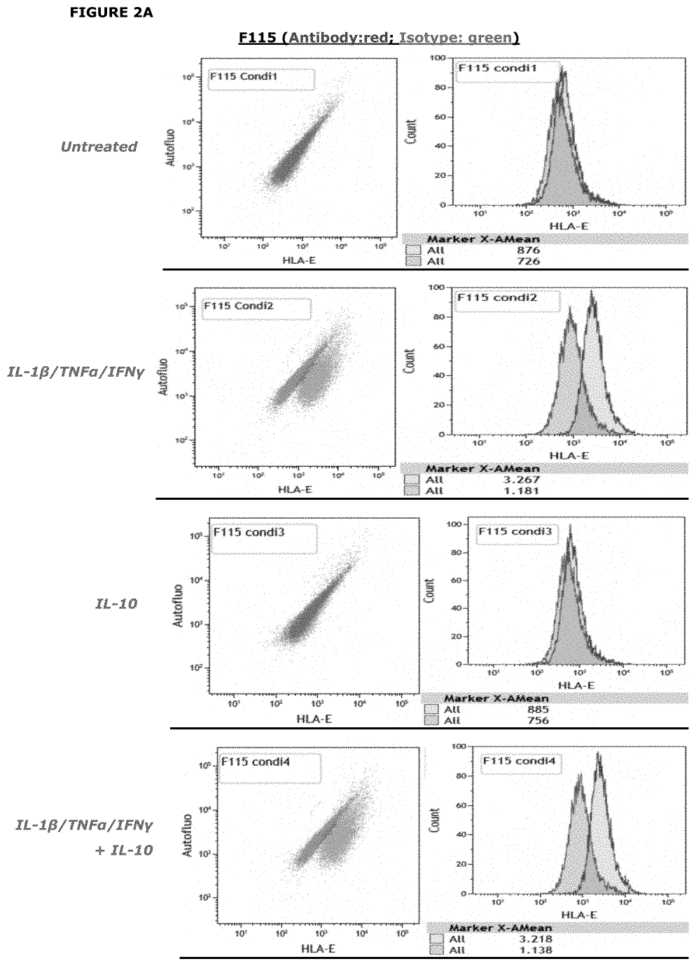

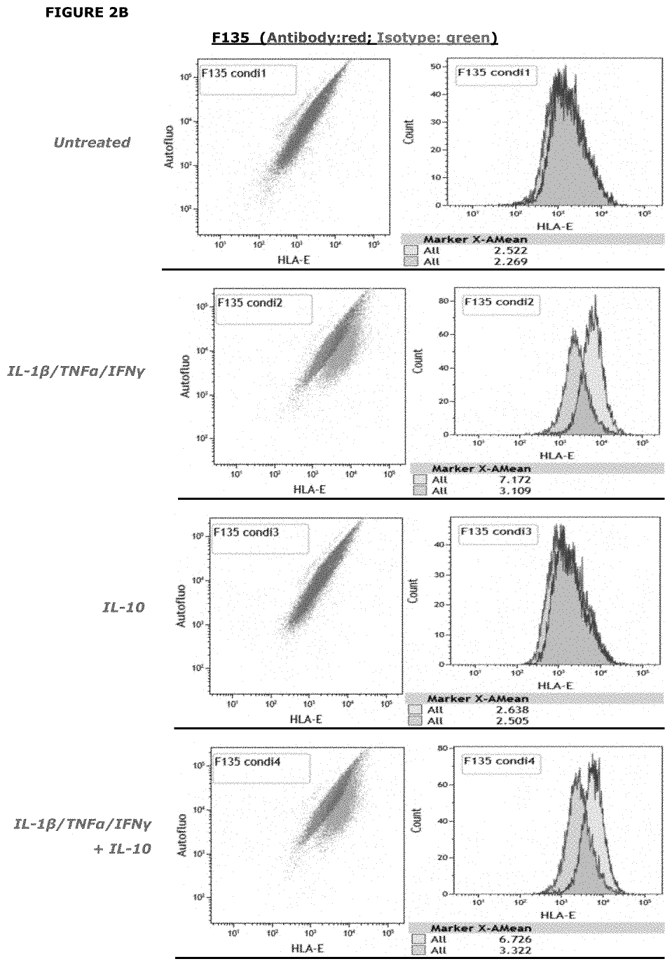

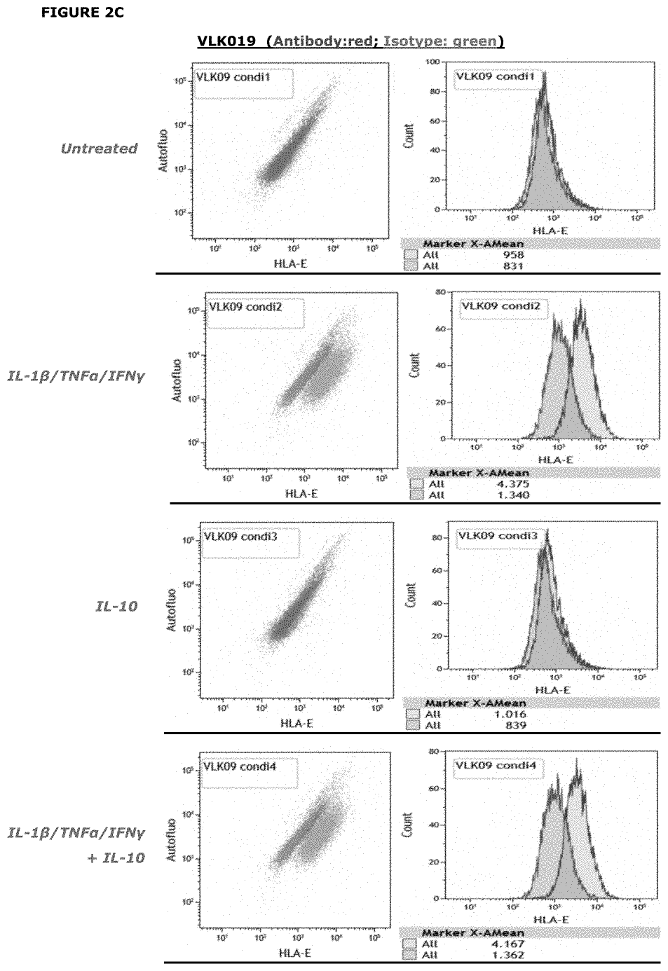

As shown in FIG. 1 and FIG. 2, preconditioning of liver progenitor cells by addition of one or more cytokines to the cell medium of the cells according to an embodiment of the invention results in cells with elevated levels of HLA-E expression as compared to a basal HLA-E expression level in non-preconditioned cells (control condition).

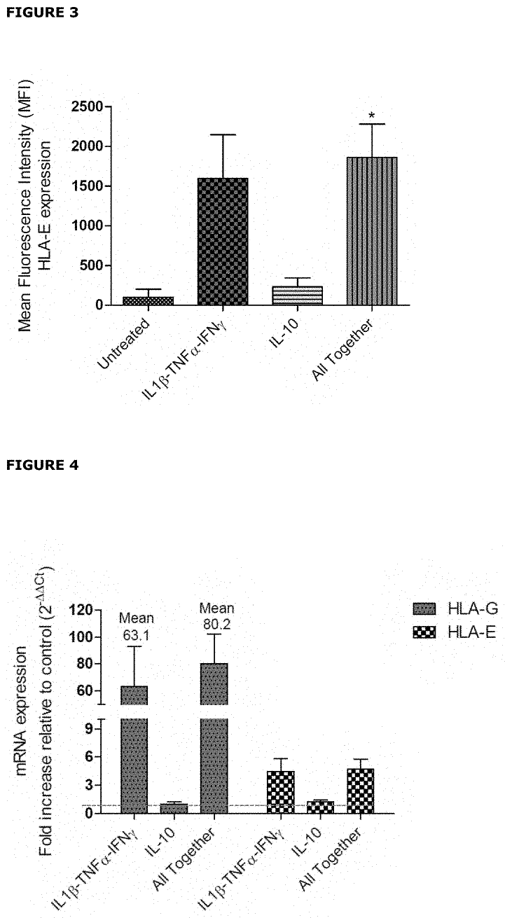

The results presented in FIG. 3 show the Mean Fluorescence Intensity (MFI) of cells stained with specific mAb and confirms the HLA-E positive identity of the preconditioned liver progenitor cell population which shows elevated levels of MFI as compared to the control condition (untreated). Results are presented as mean±SEM. All data were analysed by Prism 5 software.

### 3. Quantification of Gene Expression by Real-Time Reverse Transcription-Polymerase Chain Reaction (RT-PCR)

Preconditioned adult liver progenitor cells and control cells were prepared as described in Example 1.

Gene expression was characterized using RT-PCR. cDNA was synthesized using a commercial kit according to the manufacturer's instructions. Samples were run in duplicate on a Real-Time PCR machine. Relative quantification of gene expression was established by normalizing the signal intensity against endogenous control transcripts. After normalization, the data were plotted and compared among liver progenitor cell populations preconditioned with different cytokine combinations.

More in detail, cells were recovered for total RNA extraction using the GenElute™ Mammalian Total RNA Miniprep Kit (Sigma—RTN070). During RNA extraction, DNAse treatment was carried out using On-Column DNase I Digestion Set (Sigma ref: DNASE70). RNA was then quantified using a NanoDrop (Thermo Scientific). First-strand cDNA was synthesized using a Transcriptor First Strand cDNA Synthesis Kit (Roche—04897030001) according to the manufacturer's instructions. PCR amplification mixtures (20 μl final volume) containing template cDNA, Taqman Master Mix buffer (10 μL; Applied Biosystems), and forward and reverse primer PCRs were run in duplicate and performed on a ViiA 7 Real-Time PCR System (Applied Biosystems). The cycling conditions comprised 10 min polymerase activation at 95° C., 40 cycles at 95° C. for 15 s and 60° C. for 1 min. Quantification was normalized against the house keeping gene GAPDH.

Commercial primers and probes used for the current study are listed hereafter (all products from Thermo Fisher): GAPDH—Hs99999905_m1, HLA-G—Hs00365950_g1, HLA-E—Hs03045171_m1, IDO1—Hs00984148_m1, HGF—Hs00300159_m1, PTGS2—Hs00153133_m1, IL-6—Hs00174131_m1, IL-10—Hs00961622_m1.

FIG. 4 shows that the preconditioned liver progenitor cells are positive for HLA-E. The preconditioned liver progenitor cells have elevated levels of HLA-E expression as compared to a basal HLA-E expression in non-preconditioned liver progenitor cells. In addition, FIG. 4 shows that preconditioned HLA-E positive cells can further be positive for HLA-G. Results are expressed as mean±SEM (n=3). All data were analyzed by Prism 5 software.

Liver progenitor cells which are HLA-E positive or which have elevated levels of HLA-E can further also have elevated levels of gene expression of one or more cytokines and/or factors with immunomodulatory properties as compared to the basal expression level detected in a non-preconditioned liver progenitor or stem cell (control). FIG. 5 shows that indoleamine 2,3-dioxygenase 1 (IDO1), prostaglandin-endoperoxide synthase 2 (PTGS2), IL-6 and IL-10 gene expression levels were elevated in a population of preconditioned liver progenitor cells comprising an elevated amount of HLA-E positive cells. Tests were performed on liver progenitor cells or stem cells isolated from three different donors (n=3). Results are expressed as mean±SEM.

### 4. HLA-G Secretion: Quantification of Soluble HLA-G by CLIA (Chemiluminescent Sandwich Principle) in a Population Comprising HLA-E Positive Liver Progenitor or Stem Cells

Preconditioned liver progenitor cells were prepared as described in Example 1.

Expression of soluble proteins was measured in the supernatants harvested from the preconditioned liver progenitor cells. Soluble HLA-G protein quantification was performed using a commercial enzyme-linked immunoassay (ELISA) kit as described hereafter.

Soluble HLA-G ELISA was performed using a CLIA kit: Human HLA-G ELISA Kit (CLIA)—LS-F30041. 100 μL of cell culture supernatants or standards were incubated with the capture antibody for 90 minutes at 37° C. After incubation, 100 μl of Biotinylated Detection Antibody was added in each well for 1 hour at 37° C. Following a washing step, 100 μL/w of HRP-conjugated was incubated at 37° C. for 30 min. After five washes, the plate was incubated with a chemiluminescent substrate for 5 min at 37° C., protected from light. At the end of the incubation time, the plate was immediately analyzed to determine the Relative Light Units (RLU) of each well using a microplate luminometer. The assay's detection range was 0.16-10 ng/ml of sHLA-G. Each sample was tested in duplicate.

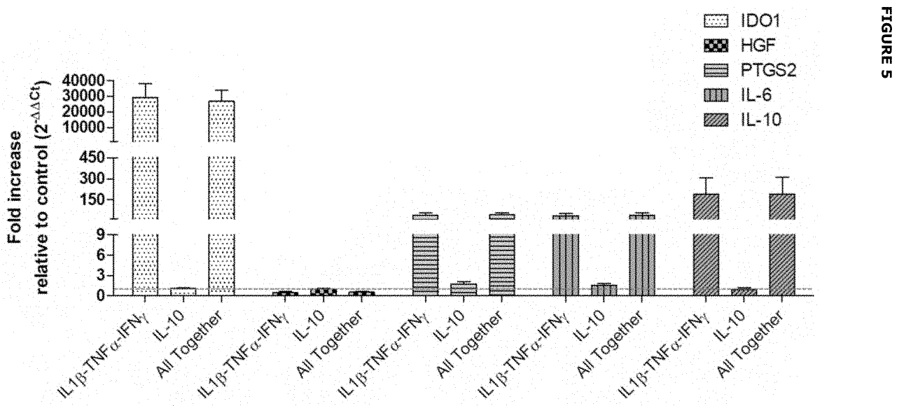

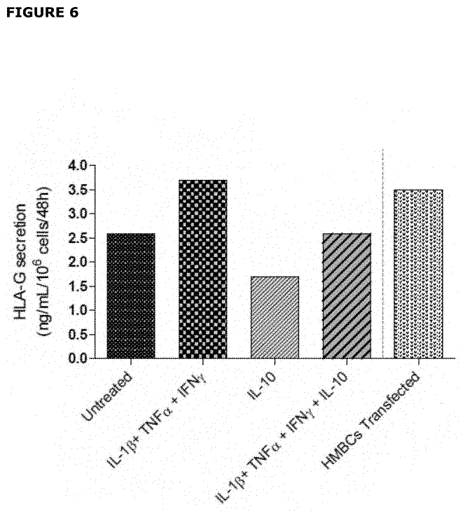

The results presented in FIG. 6 show that the preconditioned liver progenitor cells have enhanced levels of HLA-G in their supernatant.

5. Expression of Immunomodulatory Cytokines and/or Immunomodulatory Factors in HLA-E Positive Liver Progenitor or Stem Cells

Preconditioned HLA-E positive liver progenitor cells were prepared as described in Example 1.

Expression of soluble proteins was measured using a commercial enzyme-linked immunoassay (ELISA) kit. The HLA-E positive liver progenitor cells or liver progenitor cells having elevated levels of HLA-E expression as compared to a basal HLA-E expression further have elevated levels of secretion of prostaglandin E2 (PGE2) as compared to the basal level of PGE2 detected in non-preconditioned liver-derived progenitor or stem cells (untreated).

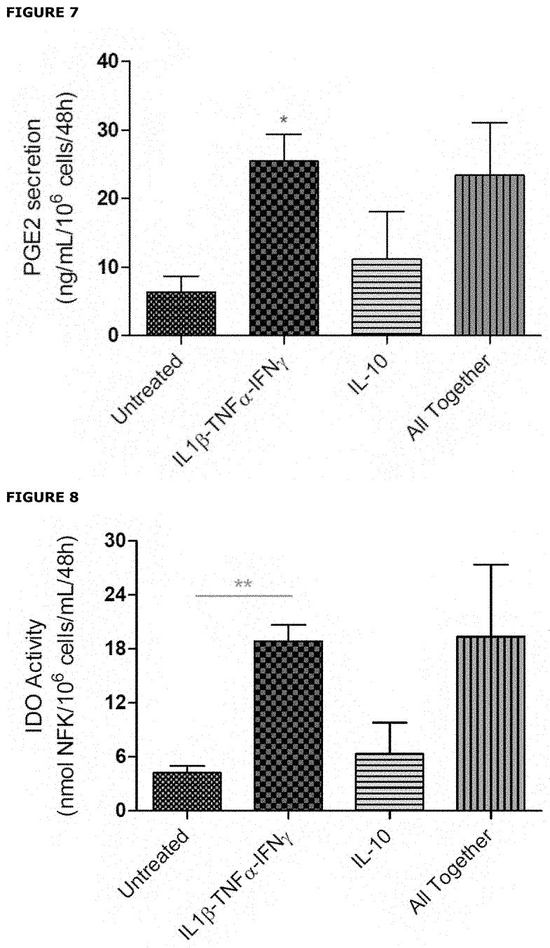

FIG. 7 confirms elevated secretion of immunomodulatory factor PGE2 in liver progenitor cells having elevated levels of HLA-E after preconditioning with one or more cytokines selected from IFNγ, TNFα, IL-1β and/or IL-10, according to embodiments of the invention. Test performed on liver progenitor cells isolated from three different donors (n=3). Results are expressed as mean±SEM. All data were analyzed by Prism 5 software.

### 6. IDO1 Activity is Enhanced in HLA-E Adult Liver Progenitor Cells According to an Embodiment of the Invention

Preconditioned HLA-E positive liver progenitor cells were prepared as described in Example 1.

The enzymatic activity of IDO1 was measured using a fluorogenic developer that selectively reacts with the compound N-formylkynurenine (NFK) to produce a highly fluorescent product (Ex/Em=402/488 nm). The formation of NFK is directly related to the amount of IDO enzyme activity.

As shown in FIG. 8, the enzymatic activity of IDO1 is enhanced in preconditioned liver progenitor cells as compared to the basal enzymatic activity of IDO1 in non-preconditioned liver progenitor cells (untreated). Test performed on liver progenitor cells isolated from three different donors (n=3). Results are expressed as mean±SEM. All data were analyzed by Prism 5 software.

It is supposed that the present invention is not restricted to any form of realization described previously and that some modifications can be added to the presented example of fabrication without reappraisal of the appended claims.

### 7. Quantification of HLA-E Expression in Human Liver Progenitor Cells

Cells were thawed and plated at 30.000 cells/cm² on cell bind surface in DMEM containing 9% FBS. The following day, cells were incubated with DMEM+9% FBS (=unstimulated condition) or DMEM+9% FBS supplemented with a pro-inflammatory cocktail consisting of IFNγ 10 ng/ml, TNFα 50 ng/ml, IL-1β 20 ng/ml (=stimulated condition). After 24 hours of incubation, cells are detached and HLA-E expression was determined by flow cytometry.

HLA-E expression was determined by flow cytometry (MacsQuant) using an anti-HLA-E antibody (#130-117-401, Clone REA1031 from Miltenyi). Relative median fluorescence intensity (rMFI, MFI antibody/MFI isotype) as well as percentage of cells expressing HLA-E has been determined using an isotype control antibody.

### Results:

HLA-E expression per cell was found to be increased in the stimulated cells versus non-stimulated cells. In addition, the percentage of HLA-E positive cells in the population was increased significantly, resulting in a population of cells with high percentage of HLA-E expressing cells, useful to be used in therapy.

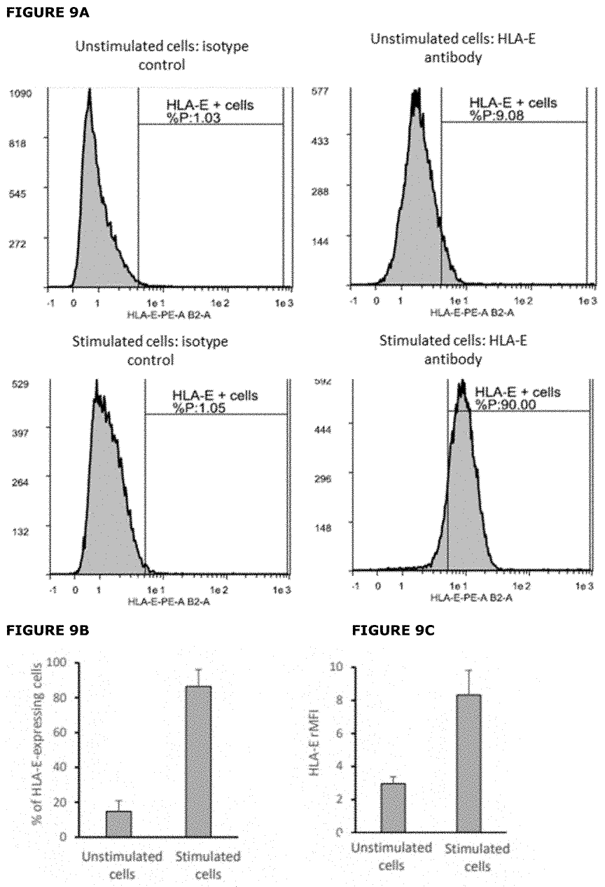

FIG. 9A shows representative histograms showing higher expression of HLA-E in liver progenitor cells stimulated with a pro-inflammatory cocktail. FIG. 9B shows the mean percentage of HLA-E positive cells with or without pro-inflammatory conditioning. After 24 hours of conditioning, the conditioned population showed an increase in HLA-E positive cells compared to the non-conditioned cells (14.7% versus 86.4%). In the stimulated condition a minimum of 69.6% and maximum of 98.2% where HLA-E positive cells, whereas in the unstimulated population, the HLA-E positive cells ranged from 4.1% to 22.3%. Cells are considered HLA-E positive when they have a higher fluorescence intensity compared to the isotype control as shown in FIG. 9A.

FIG. 9C shows an increased expression of HLA-E after 24 h of stimulation: average rMFI for stimulated cells is 8.3 compared to 3.0 for unstimulated cells. In stimulated condition an rMFI of between 6.8 and 11.8 was observed whereas in unstimulated cells an rMFI from 2.2 to 3.8 was detected. (rMFI: relative median fluorescence intensity or MFI antibody/MFI isotype (fluorescent signal when cells are stained with the HLA-E antibody divided by fluorescent signal when cells are stained with the isotype control). (n=5 including two different liver donors).

### 8. HLA-E Expression Reduces Killing of Liver Stem or Progenitor Cells by NK or CD8+ Cells

A cell-based assay was developed using CD8+ T cells isolated from PBMC to investigate the impact of HLA-E expression on liver stem or progenitor cells.

### Isolation and Culture of Human CD8+ T Cells:

Human peripheral blood mononuclear cells (PBMC) were isolated from buffy coats of normal adult donors by centrifugation on a Ficoll-Hypaque density gradient. CD8+ T cells were isolated using Dynabeads®Untouched™ Humand CD8 T cells kit according to manufacturer's instructions (Invitrogen™). Purity and viability of isolated cells has been always determined by flow cytometric analysis (APC-conjugated CD8 antibody and DAPI), showing >80% of CD8+ cells.

### Liver Stem Cell Culture and Pre-Conditioning:

Two days before the beginning of the cytotoxicity assay (=day−2), liver stem cells were thawed and plated at 30.000 cells/cm² on cell bind surface in DMEM containing 9% FBS. Next day cells were incubated with DMEM+9% FBS (=unstimulated condition) or DMEM+9% FBS supplemented with a pro-inflammatory cocktail consisting of IFNγ 10 ng/ml, TNFα 50 ng/ml, IL-1β 20 ng/ml (=stimulated condition). After 24 hours of pre-conditioning, cells were trypsinised, counted and used for cytotoxic assay or characterization (HLA-E expression by flow cytometry). HLA-E expression was checked by flow cytometry. Relative median fluorescence intensity (RMFI, MFI antibody/MFI isotype) as well as percentage of cells expressing HLA-E has been determined using an isotype control antibody.

### CD8+ T Cell Cytotoxic Assay:

Two consecutive co-cultures were performed. CD8+ T cells from the first contact were put back in culture with the same batch of liver stem cells. First contact on Day 0 started with the plating of unstimulated or stimulated cells in 24-wells plate at 20 000 cells/cm² in X-VIVO Medium. After 4 hours of incubation (37° C., 5% CO2 in humidified atmosphere), the plating of cells was checked and 2 million CD8+ T cells were added in each well with a final volume of 1 ml X-VIVO. Control conditions consisted in CD8+ T cells cultured alone or with Human T-activator anti-CD3/CD28 Dynabeads (according to manufacturer protocol). On Day 4 of the co-culture 100 UI/ml of IL-2 was added to each well. After 7 days of co-culture, 1st contact was ended by harvesting supernatant containing CD8+ T cells. Culture supernatant were kept at −80° C. for further ELISA assays while CD8+ T cells were resuspended in X-VIVO containing IL-2 and counted with flow cytometry (Absolute counting on CD8+ cells). CD8+ T cells were put back in culture for the second contact with the same condition of liver stem cells (stimulated or not) as for the first contact. Second contact lasted 5 days (=day 11) after which co-culture supernatants, CD8+ T cells and liver stem cells were harvested. Viability and cell count of CD8+ T cells and liver stem cells was assessed by flow cytometry using APC conjugated CD8 antibody and DAPI.

Effect of pro-inflammatory pre-conditioning on resistance to CD8+ T cell cytotoxicity was assessed by optical microscopy, determination of viability and cell count at the end of the co-culture as well as by measuring secreted IFNγ (surrogate of CD8+ T cell activation) in co-culture supernatant.

### Results:

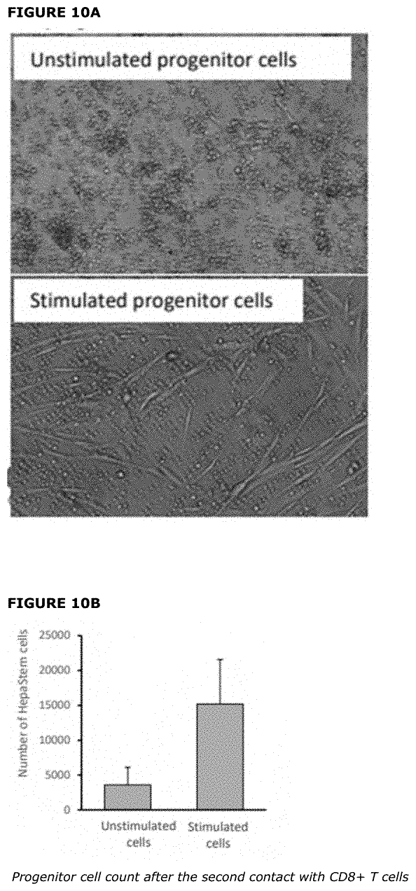

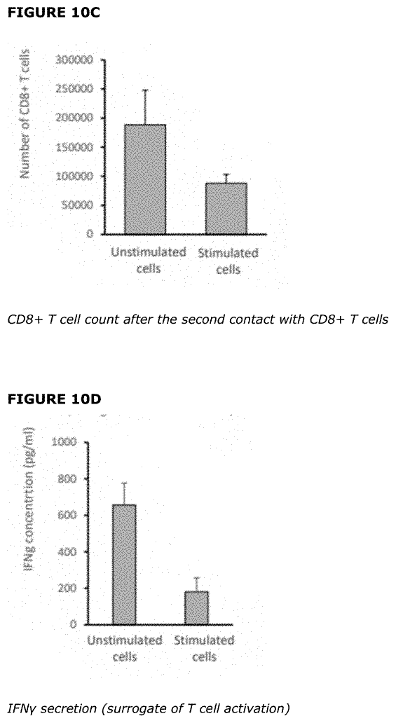

FIG. 10A show representative pictures of liver stem cells after co-culture with CD8+ T cells (day 5 of the second contact). Liver stem cells stimulated with pro-inflammatory cytokines are still observed at the end of the co-culture while most the unstimulated cells are killed.

FIG. 10B shows the liver stem cell count at the end of the co-culture with CD8+ T cells (second contact). Cell count was determined by flow cytometry (absolute count with the MacsQuant from Miltenyi, excluding CD8+ positive cells). More cells are counted when cells are stimulated compared to unstimulated cells, with an average of 15199 compared to 3581 cells, respectively (n=4 including two different liver donors and two PBMC donors).

FIG. 10C shows CD8+ T cell count at the end of the co-culture with liver stem cells (second contact). The number of CD8+ T cells was measured at the end of the experiment because T cells proliferate once activated. On average, there is more CD8+ T cells at the end of the co-culture with unstimulated cells compared to with stimulated cells (188369 vs 87930 cells). The number of CD8+ T cell is counted by flow cytometry (absolute count with the MacsQuant from Miltenyi, including only CD8+ positive cells). (n=4 including two different liver donors and two PBMC donors.)

FIG. 10D shows IFNγ concentration at the end of the liver progenitor cell population expressing high level of HLA-E/CD8+ T cells co-culture (second contact). The concentration of IFNγ in the supernatant of the co-culture is higher when CD8+ T cells are incubated with unstimulated liver progenitor cells compared to stimulated liver progenitor cells. As IFNγ secretion by T cell is linked to activation, this shows that stimulated liver stem cells activate less CD8+ T cells. IFNγ was measured by ELISA in CD8+ T cell—liver stem cell co-culture supernatants. (n=12 including two different liver stem cell donors and four PBMC donors)

### CONCLUSION

After stimulation with a pro-inflammatory cocktail, liver progenitor cell population expressing high level of HLA-E are protected from CD8+ T cell-mediated cytolysis. In addition, activation of CD8+ T cells is reduced when incubated with stimulated liver stem cells compared to unstimulated cells (as shown by reduce proliferation of T cells and IFNγ secretion. This protection correlates with a higher expression level of HLA-E.

1. A composition comprising human adult liver-derived progenitor or stem cells characterized in that at least 60% of said progenitor or stem cells express cell surface marker HLA-E.

2. Composition according to claim 1, wherein at least 75% of said progenitor or stem cells express cell surface marker HLA-E.

3. Composition according to claim 1, wherein the HLA-E expressing cells have an HLA-E relative median fluorescence intensity (rMFI) of at least 6.5 as measured by flow cytometry and using an anti-human HLA-E antibody.

4. Composition according to claim 1, wherein the HLA-E expressing cells exhibit an HLA-E relative medium fluorescence intensity (rMFI) of at least 7 as measured by flow cytometry.

5. Composition according to claim 1, wherein said human adult-liver-derived progenitor or stem cells are positive for at least one mesenchymal marker selected from Vimentin, CD13, CD90, CD73, CD44, CD29, a-smooth muscle actin (ASMA), and/or CD140b, or a combination thereof.

6. Composition according to claim 1, wherein said composition comprises secreted HLA-G.

7. The composition according to claim 1, characterized in that said composition further comprises a pharmaceutically acceptable carrier.

8. An isolated liver progenitor cell or stem cell which is HLA-E positive.

9. The isolated liver progenitor cell or stem cell according to claim 8, wherein said cell is a human liver cell, preferably an adult human liver cell.

10. The isolated liver progenitor cell or stem cell according to claim 8, wherein the HLA-E expressing cells have an HLA-E relative median fluorescence intensity (rMFI) of at least 6.5 as measured by flow cytometry and using an anti-human HLA-E antibody.

11. The isolated liver progenitor cell or stem cell according to claim 8, characterized in that said cell is preconditioned with one or more cytokines.

12. The isolated liver progenitor cell or stem cell according to claim 11, characterized in that said cell has elevated levels of HLA-E expression as compared to a basal HLA-E expression in a non-preconditioned liver progenitor or stem cell.

13. The isolated liver progenitor cell or stem cell according to claim 11, characterized in that said cytokines are selected from IFNγ, TNFα, IL-lb, IL-10 and any combinations thereof.

14. The isolated liver progenitor cell or stem cell according to claim 11, characterized in that said cytokines are added to the cell medium of said cell at a total concentration of between 5 ng/ml and 400 ng/ml.

15. The isolated liver progenitor cell or stem cell according to claim 11, characterized in that the individual cytokine concentration in the cell medium is between 5 ng/ml and 100 ng/ml.

16. The isolated liver progenitor cell or stem cell according to claim 11, characterized in that said cytokines are at least IFNy and TNFa.

17. The isolated liver progenitor cell or stem cell according to claim 16, wherein said cytokines further comprise IL-lb.

18. The isolated liver progenitor cell or stem cell according to claim 11, characterized in that said cell is preconditioned for 1 hour to 72 hours.

19. (canceled)

20. The isolated liver progenitor cell or stem cell according to claim 11, characterized in that said cell further has elevated levels of PGE2 secretion, as compared to a basal PGE2 secretion in a non-preconditioned liver progenitor or stem cell; and/or elevated levels of IDO enzymatic activity, as compared to a basal IDO enzymatic activity in a non-preconditioned liver progenitor or stem cell.

21. A cell population comprising isolated liver progenitor cells which are HLA-E positive.

22.-33. (canceled)
